(19) United States
(12) Patent Application Publication
(10) Pub. No.: US 2023/0284994 A1
(43) Pub. Date: Sep. 14, 2023

(54) PET-CT IMAGING METHODS, CONTRAST AGENTS AND PHARMACEUTICAL COMPOSITIONS FOR USE IN SAID IMAGING METHODS

(71) Applicant: ADIPOSS SÁRL, Plan-les-Ouates (CH)

(72) Inventors: Nathalie STRANSKY-HEILKRON, Valserhône (FR); Andrej BABIC, Veigy-Foncenex (FR)

(21) Appl. No.: 18/019,752

(22) PCT Filed: Aug. 6, 2021
(86) PCT No.: PCT/EP2021/072014

§ 371 Date: Feb. 3, 2023

(30) Foreign Application Priority Data
Aug. 6, 2020 (EP) 20189928.3

(51) Int. Cl. A61B 6/00; A61K 49/04; A61B 6/03

(52) U.S. Cl. CPC A61B 6/5235; A61K 49/0438; A61K 49/0461; A61B 6/032; A61B 6/037

(57) ABSTRACT

The invention describes a method to differentiate tumour tissue from brown adipose tissue on a PET-CT scan. In particular, the invention concerns a method for discriminating primary tumour and/or metastases from brown and/or beige adipose tissue, by PET-CT imaging, in a human cancer patient.

### FIELD OF THE INVENTION

The invention describes a method to differentiate tumour tissue from brown adipose tissue on a PET-CT scan with a medical imaging agent. In particular, the invention concerns a method for discriminating primary tumour and/or metastases from brown and/or beige adipose tissue, by PET-CT imaging, in a human cancer patient. Also described is a prognostic method for cancer evaluation by assessing the quantity of activated brown and/or beige tissue, by PET-CT imaging, in a human cancer patient.

### BACKGROUND OF THE INVENTION

Positron emission tomography (PET) is an imaging technique that uses radioactive substances to visualize and measure metabolic processes in the body. PET is mainly used in the area of medical imaging for detecting or measuring changes in physiological activities like metabolism, blood flow, regional chemical composition, and absorption.

PET scan requires the administration of a radioactive tracer to the patient before performing the scan. The most commonly used PET tracer is ¹⁸FDG (2-[fluorine-18]fluoro-2-deoxy-D-glucose). This tracer is used to identify the increased glycolytic activity in tissues, especially malignant cells, in which glucose is preferentially concentrated due to an increase in membrane glucose transporters as well as to an increase in some of the principal enzymes, such as hexokinase, responsible for phosphorylation of glucose. ¹⁸FDG is transported into tumour cells, similarly to glucose, by means of glucose transporter proteins known as GLUT transporters and subsequently phosphorylated by hexokinase to ¹⁸FDG 6-phosphate. ¹⁸FDG 6-phosphate is not efficiently metabolized further and therefore accumulates within the cell. This process of “metabolic trapping” of ¹⁸FDG in the cell constitutes the basis for imaging the in vivo distribution of the tracer with ¹⁸FDG PET. The concentrations of imaged ¹⁸FDG tracer indicate tissue metabolic activity as it corresponds to the regional glucose uptake. ¹⁸FDG is used to explore the possibility of cancer spreading to other body sites (cancer metastases). These ¹⁸FDG PET scans for detecting primary cancer and cancer metastases are the most common in standard medical care (representing 90% of current PET scans). It is possible to image the entire body in a single session, increasing the opportunity for finding unsuspected disease sites.

On the other hand, computed tomography (CT) is a medical imaging procedure that uses computer-processed combinations of several X-ray measurements taken from different angles to produce cross-sectional (tomographic) X-ray images of specific areas of a scanned object.

PET-CT combines a PET scanner and a CT scanner to acquire images from both devices in the same (single) scanning session, which are combined into a single image. Thus, PET-CT allows precise anatomical alignement of functional imaging obtained by PET.

¹⁸FDG PET-CT scan is used for the diagnosis, and/or staging, and/or re-staging, and/or assessment of disease dissemination (metastases) and/or therapy response of the following malignancies: lung, colorectal, breast, gynaecological, head and neck, oesophageal, gastric, biliary tract, follicular and medullar thyroid, and pancreatic cancers, melanomas, lymphomas, multiple myelomas, sarcomas, primary brain tumours and other tumours¹.

Brown adipose tissue (BAT) is responsible for cold- and food-induced non-shivering thermogenesis. It is also responsible for increased energy expenditure in patients with cancer and chronic diseases. ¹⁸FDG can be taken up in brown adipose tissue in the neck (cervical), shoulder (supraclavicular), paravertebral region, mediastinal, perirenal, and perigastric region. Other non-classical areas of uptake have also been observed.

Beige adipose tissue is another type of thermogenic adipose tissue that arises in white adipose depots in response to physio-pathological stimuli. It has similar function as brown adipose tissue.

While being very sensitive to identifying sites of malignancy, ¹⁸FDG PET is not very cancer tissue-specific. False positive results may be observed with benign diseases, with a reported false positive rate of 13% and false negative rate of 9%². These false-positive areas of metabolic activity have the potential for significant morbidity and mortality if not accurately recognized³. Brown adipose tissue uptake of ¹⁸FDG has been reported to be observed in 5 to 10% of patients^{4,5}. It is especially commonly observed in women and children during cold weather months. In particular, Hong et al.⁶ describe frequent uptake of ¹⁸FDG in brown adipose tissue (BAT) in children. In children, ¹⁸FDG uptake has been reported in up to 50% of children having a ¹⁸FDG PET scan. Hong at al. ⁶ also describe the parameters that influence the uptake of ¹⁸FDG in the brown fat (for example: age, gender, environmental temperature . . . ). Further, they describe the current methods to reduce ¹⁸FDG uptake in BAT: pharmacological approaches (opiates and benzodiazepines), diet (high-fat and low-carbohydrate diet the night before the scanning procedure) and warming prior to and after the injection of ¹⁸FDG.

Steinberg et al.⁷ also describe the uptake of ¹⁸FDG in BAT on PET-CT scans in some patients and the factors that influence the said uptake. The proportion of ¹⁸FDG PET-CT scans with ¹⁸FDG uptake in the BAT can reach 17% in patients with lymphoma and 17 to 80% of patients with breast cancer. In 15% of ¹⁸FDG PET-CT scan with tracer uptake in the brown adipose tissue, the nuclear medicine physician is unable to assess the scan due to uncertainty. According to the authors⁷, it is unknown why BAT is activated in some patients but not in others, or why the ¹⁸FDG uptake in BAT varies also within patients. The authors also explain that ¹⁸FDG uptake in BAT is a confounding factor that can lead to erroneous conclusions (false positives or false negatives). They describe the patients' characteristics that make it more likely to have ¹⁸FDG uptake in BAT (sex, age, BMI). Finally they cite outdoor temperature and time of the scanning procedure as influencing factors.

Cohade et al.⁸ also describe the problem that has to be solved (¹⁸FDG uptake in BAT and detection by PET-CT) and influencing parameters such as age, sex, BMI.

¹⁸FDG uptake in hyper-metabolic brown adipose tissues is well recognized as a potential source of false positive in ¹⁸FDG PET-CT imaging^{3,9}. Indeed, Long et al.⁹ describe the risk of false positive and false negatives on an ¹⁸FDG PET-CT scan. They strongly advise the physicians to use the CT scan to help reach a conclusion (tumor or BAT uptake). They suggest some pharmacological approaches as a solution (administration of propranolol or keeping the patients warm) to prevent BAT activation and thus ¹⁸FDG accumulation in BAT.

Wang et al.¹⁰ also describe one of the problems that have to be solved, i.e. discriminating between ¹⁸FDG uptake in BAT and in tumor tissues. The oral CT contrast agents described in this article delineate the digestive tract only, with no systemic uptake of the oral contrast agents in this case. This article also states that benzodiazepines (diazepam) can be used to block ¹⁸FDG uptake in BAT.

Pharmacological methods can be used to decrease the uptake of ¹⁸FDG in the brown adipose tissue, but the patients are then at higher risk of adverse reactions. Furthermore, pharmacological treatment prior to scan is not advised and has to be used with precautions in the paediatric population. Reduction of ¹⁸FDG uptake is usually attempted only by keeping the patients warm and providing them with blankets during the uptake phase (between ¹⁸FDG injection and PET scan). The current method to discriminate metastases from brown adipose tissue on a PET scan is by comparing the result of the PET scan with the detailed and time-consuming analysis of axial slices of the CT scan by the nuclear medicine physician and radiologist, respectively. If the area of ¹⁸FDG uptake corresponds to fat attenuation on the CT scan, the tracer uptake is then attributed to brown adipose tissue activation.

WO 2008/153928 A2 ¹¹ (KOLODNY GERALD [US], WILLIAMS GETHIN [US]) describes a method to decrease ¹⁸FDG uptake in BAT. It involves following a high fat, no carbohydrates, low protein diet several hours before the ¹⁸FDG PET-CT scan to reduce the FDG uptake in BAT (and myocardium).

WO 2019/030024 A1¹² (UNIV GENEVE [CH]) describes an iodinated CT contrast agent made of fatty acid derivatives for non-invasive visualisation and quantification of the brown and beige adipose tissue. One of the distinctive features of this contrast agent is to be given per oral route to provide contrast in brown adipose tissue. Brown adipose tissue is then detected by CT scan. This CT contrast agent is used in the context of a CT scan to detect brown adipose tissue only.

In cancer patients with high brown adipose tissue activity and/or volume and/or surface characterized by the uptake of ¹⁸FDG, worse outcomes are observed¹³. In these patients, more metabolically active brown adipose tissue (characterized by ¹⁸FDG uptake) was associated with more active neoplastic status. In particular, Huang et al.³ stated that neoplastic status is a critical determinant of BAT activity in patients living in the tropics, where the influence of outdoor temperature on BAT activation is minimal. However, the authors based their conclusion on 30 out of 1740 patients having had an ¹⁸FDG PET-CT scan (37 ¹⁸FDG PET-CT scans positive for BAT out of 1903). BAT activity was quantified on the ¹⁸FDG PET-CT scan. Out of 1171 patients with cancer history, only 21 (1.8%) showed ¹⁸FDG accumulation in BAT. From these results, it can be concluded that ¹⁸FDG PET-CT scan clearly lacks sensitivity to detect BAT. The estimated sensitivity of standard ¹⁸FDG PET-CT scan for BAT is between 1.8 and 10% today.

Abnormal BAT volume and/or surface and/or activity has been associated with an increased likelihood of tumour recurrence and/or tumour-associated mortality¹⁴. This may be linked not only to tumour progression and/or severity (inflammatory and tumour factor promoting the activation of brown adipose tissue and browning of white adipose tissue) but also the development of body wasting—cachexia. Indeed, Chu et al.¹⁴ link BAT-positive ¹⁸FDG PET scans to cancer progression and cancer-associated mortality. However, the results that are presented are based on 132 patients who had BAT-positive ¹⁸FDG PET scans. It is not known how many cancer patients had BAT-negative ¹⁸FDG PET scans, but the authors themselves point the lack of sensitivity of ¹⁸FDG PET scan to assess presence and/or activity of BAT.

Bos et al.¹⁵ found that BAT-positive ¹⁸FDG PET scans were significantly more associated with patients having active cancer than with patients without active cancer. However, once again, the sample size was very small (142 patients out of 21262, i.e. 0.66%), pointing to the lack of sensitivity of ¹⁸FDG PET scan to detect and quantify BAT under standard PET scan conditions.

Abnormal BAT volume and/or surface and/or activity could be increased or decreased compared to BAT volume and/or surface in a healthy subject. Today, the staging of a tumour and the prognosis of the cancer is usually based on the TNM staging system. It is a classification system of the anatomical extent of tumours.

TNM is a notation system that describes the stage of a cancer, which originates from a solid tumour, using alphanumeric codes:
T describes the size of the primary tumour and whether it has invaded nearby tissue,
N describes nearby (regional) lymph nodes that are involved,
M describes distant metastases.

Although this staging system can give some indication about the outcome of the cancer at the time of diagnosis (the higher the cancer grade, the poorer its prognosis), it does not give any indication about the aggressiveness of the cancer.

Unfortunately, there is currently no validated method to make sure that ¹⁸FDG uptake in some anatomical areas is due to metabolically active brown adipose tissue and not to tumours or metastases. In some cases, when the nuclear medicine physician and the radiologist cannot reach a meaningful conclusion, the ¹⁸FDG PET-CT scan has to be repeated, resulting in additional exposure of the patient to radiation, an increase in the time spent in the hospital setting for the patient and additional cost for the healthcare system and/or patient.

### BRIEF DESCRIPTION OF THE INVENTION

The present invention relates to means and methods for overcoming the deficiencies of the prior art discussed above. In particular, the present invention provides various methods, contrast agents for use and pharmaceutical compositions for use, which are allow to increase accuracy of determinations based on imaging methods involving the use of a PET-CT contrast agent and a PET tracer such as ¹⁸FDG. These methods of the invention, contrast agents for use according to the invention and pharmaceutical compositions for use according to the invention are specified in the appended claims and the numbered items of the special embodiment described hereinbelow.

Further information on the means and methods of the invention is given in the detailed description below. In more detail, the present invention provides inter alia a method for discriminating ¹⁸FDG uptake in metastases and/or tumours from brown adipose tissue on a ¹⁸FDG PET-CT scan. It consists in giving the patient a PET-CT contrast agent that specifically accumulates in the brown adipose tissue prior to the ¹⁸FDG PET-CT scan. The PET-CT contrast agent is preferably given to the patient per oral route but could also be administered by other routes (intravenous, intrathecal, intralymphatic, intra-arterial, intraperitoneal, subcutaneous). In some embodiments, colocalized signals from the PET tracer (¹⁸FDG) and the PET-CT contrast agent may be attributed to the uptake of ¹⁸FDG in the brown adipose tissue.

Contrary to Wang et al.¹⁰, the present invention does not aim at reducing ¹⁸FDG uptake in BAT but at using a BAT-specific medical imaging agent to discriminate between ¹⁸FDG uptake in tumor tissue and in BAT on ¹⁸FDG PET-CT scans.

One of the objects of the invention is to provide a method for discriminating primary tumour and/or metastases from brown and/or beige adipose tissue in a human cancer patient by PET-CT imaging. The method comprises the steps of:
a) administering to said human cancer patient a PET-CT contrast agent, comprising an iodinated fatty acids and/or esters and/or salts and/or mixtures thereof according to general formula I:

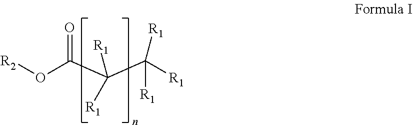

wherein n=14-16;

R₁ is H or I, with the provisions that the number of iodine atoms is 1 to 6, and that the iodine atoms are neither geminal nor vicinal;

and where R₂ is selected from H, unsaturated or saturated, linear or branched alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles. Each of these halogen substituents may be independently selected from F, Cl, Br and I. If multiple iodine atoms are present, the same proviso applies as defined herein for the R₁ groups, i.e. there must not be any two iodine atoms in geminal or vicinal position. It is also possible to rely on the preferred R₂ groups as described hereinbelow. Yet another option is to use a compound of formula A, B or C as described hereinbelow,
b) administering a ¹⁸FDG PET tracer; at least 3 hours after the administration of the PET-CT contrast agent of step a),
c) performing an ¹⁸FDG PET-CT scan to said human cancer patient.

As noted above, in some embodiments, the ¹⁸FDG PET-CT scan of step c) is used for colocalization of positive contrast enhancement of said ¹⁸FDG and said PET-CT contrast agent.

Another object of the invention is to provide a method for discriminating primary tumour and/or metastases from brown and/or beige adipose tissue in a human cancer patient by PET-CT imaging. The method comprises the steps of:
a) administering to said human cancer patient a PET-CT contrast agent, comprising an iodinated fatty acids and/or esters and/or salts and/or mixtures thereof according to general formula I:

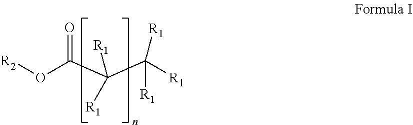

wherein n=14-16;

R₁ is H or I, with the provisions that the number of iodine atoms is 1 to 6, and that the iodine atoms are neither geminal nor vicinal;

and wherein R₂ is selected from H, unsaturated or saturated, linear or branched alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles. Each of these halogen substituents may be independently selected from F, Cl, Br and I. If multiple iodine atoms are present, the same proviso applies as defined herein for the R₁ groups, i.e. there must not be any two iodine atoms in geminal or vicinal position. It is also possible to rely on the preferred R₂ groups as described hereinbelow. Yet another option is to use a compound of formula A, B or C as described hereinbelow,
b) administering a PET tracer; at least 3 hours after the administration of the PET-CT contrast agent of step a),
c) performing aPET-CT scan to said human cancer patient.

Yet another object of the present invention is to provide a method for identifying a metabolic disease in a human subject, by PET-CT imaging brown and/or beige adipose tissue in said human subject, the method comprises the steps of:
a) administering to said human subject a PET-CT contrast agent, comprising an iodinated fatty acids and/or esters and/or salts and/or mixtures thereof according to general formula I:

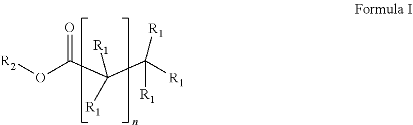

wherein n=14-16;

R₁ is H or I, with the provisions that the number of iodine atoms is 1 to 6, and that the iodine atoms are neither geminal nor vicinal;

and where R₂ is selected from H, unsaturated or saturated, linear or branched alkyls, alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles. Each of these halogen substituents may be independently selected from F, Cl, Br and I. If multiple iodine atoms are present, the same proviso applies as defined herein for the R₁ groups, i.e. there must not be any two iodine atoms in geminal or vicinal position. It is also possible to rely on the preferred R₂ groups as described hereinbelow. Yet another option is to use a compound of formula A, B or C as described hereinbelow,
b) administering a ¹⁸FDG PET tracer; at least 3 hours after the administration of the PET-CT contrast agent of step a),
c) performing an ¹⁸FDG PET-CT scan to said human subject.

In some embodiments of this method, the ¹⁸FDG PET-CT scan of step c) is used for colocalization of positive contrast enhancement of said ¹⁸FDG and said PET-CT contrast agent.

In a variant of the above method, it is possible to use a PET tracer other than ¹⁸FDG. The PET-CT scan of step c) must of course be adapted to the alternative PET tracer, but otherwise the method may be performed as outlined above.

The person skilled in the art will appreciate that the brown adipose tissue volume and/or surface and/or activity can be low or medium or high as assesed from the PET-CT scan. For example, low BAT volume could be 100 mL or less, medium BAT volume could be between 100 and 200 mL, and high BAT volume could be above 200 mL¹⁶.

The skilled artisan can then assess tumor progression and cancer prognosis on the PET-CT scan.

Uptake of the PET-CT contrast agent by brown adipose tissue, observed on the PET-CT scan, is correlated with the prognosis for the cancer patient, depending on cancer type. For most cancers, higher uptake than in healthy people means a poor prognosis and additional medical attention for this patient is required. For other malignancies, especially influenced by hormones (such as some breast cancers), higher uptake means a better cancer outcome.

Both objects of the invention (detection of malignant tissues and assessment of cancer prognosis) can be performed at the same time by a single PET-CT scan.

Other objects and advantages of the invention will become apparent to those skilled in the art from a review of the ensuing detailed description, which proceeds with reference to the following illustrative drawings, and the attendant claims.

### BRIEF DESCRIPTION OF THE FIGURES

FIG. 1: Shows a description of one of the problems to be solved by the present invention, i.e. uptake of ¹⁸FDG in brown adipose tissue that may lead to an erroneous interpretation. A: ¹⁸FDG PET scan of a 56 year-old woman with non small cell lung cancer. Black arrow: active lung cancer. White arrows: Brown adipose tissue. From Bos et al., 2019¹⁵. B: ¹⁸FDG PET scan of a 13-year-old boy with extensive ¹⁸FDG uptake by BAT in the neck and supraclavicular-axillary, paravertebral-intercostal and mediastinal regions. Arrows: uncommon contiguous, curvilinear uptake around the lateral edges of the kidneys. From Hong et al., 2011⁶. C: ¹⁸FDG PET scan of a 15 year-old girl, with asymmetric uptake of ¹⁸FDG in BAT in the upper neck (arrows).

FIG. 2: a: Axial PET-CT scans of a metastatic melanoma mouse with or without PET-CT contrast agent. Top: ¹⁸FDG PET-CT scan without PET-CT contrast agent. Bottom: ¹⁸FDG PET-CT scan with PET-CT contrast agent. Left: ¹⁸FDG PET scans. The black arrows point to ¹⁸FDG positive areas (metastases and/or BAT). Right: ¹⁸FDG PET-CT scans. The ¹⁸FDG signal in BAT can be ruled out as a metastasis (white arrow), which is confirmed by necropsy (b: organs in place, arrows=metastases; c: lungs, metastases appear black).

FIG. 3: a: Coronal ¹⁸FDG PET-CT scans of a metastatic melanoma mouse with or without PET-CT contrast agent. Top: ¹⁸FDG PET-CT scan without PET-CT contrast agent. Bottom: ¹⁸FDG PET-CT scan with PET-CT contrast agent. Left: ¹⁸FDG PET scans. The black arrows point to ¹⁸FDG positive areas (metastases and/or BAT). Right: ¹⁸FDG PET-CT scans. The ¹⁸FDG signal in BAT is easily ruled out as a metastasis (white arrow). Confirmed by necropsy (b: organs in place, arrows point to metastases; c: lungs, metastases appear black.

FIG. 4: Positive predictive value of ¹⁸FDG PET-CT scan for tumour metastases without and with the PET-CT contrast agent and method of the invention

In total, based on necropsy (standard of truth), the six mice presented 24 metastases. On the ¹⁸FDG PET-CT scans, four mice had a positive signal in BAT, that could be interpreted as false positive metastases. Without the PET-CT contrast agent and method of the invention, the positive predictive value PPV of the ¹⁸FDG PET-CT scan for metastases was 85%. By using the PET-CT contrast agent, the PPV of the ¹⁸FDG PET-CT scan for metastases was 100%. The CT contrast agent of the invention thus improves the PPV of the ¹⁸FDG PET-CT scan for tumour metastases by 15%.

FIG. 5: Sensitivity of ¹⁸FDG PET-CT scan for brown adipose tissue without and with the PET-CT contrast agent and method of the invention

In total, ¹⁸FDG uptake in BAT was present in nine out of 12 PET-CT scans. However, all mice had BAT (confirmed by necropsy). The sensitivity of the ¹⁸FDG PET-CT scan for BAT without the PET-CT contrast agent was 75%. The sensitivity for BAT was 100% using the PET-CT contrast agent. The sensitivity of the ¹⁸FDG PET-CT scan for BAT in mice was thus improved by 25% using the PET-CT contrast agent and method of the invention.

### DETAILED DESCRIPTION OF THE INVENTION

### Definitions

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control.

Unless defined otherwise, all technical and scientific terms used herein have the same meaning as is commonly understood by one of skill in art to which the subject matter herein belongs. As used herein, the following definitions are supplied in order to facilitate the understanding of the present invention.

Unless specified otherwise or the context dictates otherwise, references to the “PET-CT contrast agent” are to be understood as references to the PET-CT contrast agent as described in the corresponding sections hereinbelow.

The term “comprise” is generally used in the sense of include, that is to say permitting the presence of one or more features or components. As one specific embodiment, the term “comprise” encompasses also the meaning of the term “consist of”.

As used in the specification and claims, the singular forms “a”, “an” and “the” include plural references unless the context clearly dictates otherwise.

As used herein the terms “subject” or “patient” are well-recognized in the art and are used interchangeably herein to refer to a mammal, including dog, cat, rat, mouse, monkey, cow, horse, goat, sheep, pig, camel, and, most preferably, a human. In some embodiments, the subject is a subject in need of a diagnosis or a subject with a diagnosed disease or disorder. However, in other embodiments, the subject can be a healthy subject. The term does not denote a particular age or sex. Thus, adult and new-born subjects, whether male or female, are intended to be covered.

As used herein, the “human cancer patient” is a patient suffering from and/or having been diagnosed with any type of solid cancer and preferably cancer selected from the group consisting of lung, colorectal, breast, gynaecological, head and neck, oesophageal, gastric, biliary tract, follicular and medullar thyroid, and pancreatic cancer, leukaemia, melanoma, lymphoma, multiple myeloma, sarcoma, pheochromacytoma and primary brain tumours. In some embodiments, said human cancer patient may or may not have undergone a prior administration of ¹⁸FDG PET tracer.

As used herein, and unless indicated otherwise or the context dictates otherwise, the term “area” is intended to characterize a relevant part of the body appearing in an imaging method, the term “surface” is intended to characterize the quantification, expressed as a numerical value, of the surface area of a relevant part of the body appearing on a 2D projection of an imaging method, and the term “volume” is intended to characterize the quantification, expressed as a numerical value, of the volume of a relevant part of the body appearing in a 3D imaging method. The term “volume and/or surface” intends to refer to the volume, as defined above, in case data analysis of an imaging method is performed on 3D data while it intends to refer to the surface, as defined above, in case data analysis of an imaging method is performed on 2D data.

The term “formulation” or “pharmaceutical formulation” encompasses solid formulations such as tablets, enteric coated tablets, controlled-release tablets, sustained-release tablets, capsules and self-emulsifying pharmaceutical forms. It also encompasses liquid and semi-solid formulations such as solutions, suspensions, emulsions, topical preparations, suppositories, enemas, and parenteral formulations for injections and infusions.

The term “biocompatible” is used herein in the commonly used sense, and in particular as not being toxic.

The term “ethiodized oil”, is oil of natural origin and converted by organic synthetic procedures to ethyl esters of iodinated fatty acids to be used as injectable as a radio-opaque contrast agent that is used to outline structures in radiological investigations. Ethiodized oil is composed of iodine combined with ethyl esters of fatty acids of poppyseed oil, primarily as ethyl monoiodostearate and ethyl diiodostearate. Despite the precise structure is unknown it is comprised within the definition of formula I.

In chemistry, the term “geminal” used herein refers to the relationship between two atoms or functional groups that are attached to the same atom.

The related term “vicinal” refers to the relationship between two functional groups that are attached to adjacent atoms. Currently it is almost impossible to synthetize stable iodinated fatty acids and/or esters thereof having iodine atoms attached to adjacent carbon atoms (i.e. vicinal). Because of steric hindrance, those molecules are unstable and cannot be used for the purpose of the present invention. However, it might be possible that in the future, the skilled in the art would find a technical solution to this problem. Thus, in case stable iodinated fatty acids and/or esters there of having iodine atoms in vicinal positions are provided, it is believed that those compounds will also be suitable in solving the technical problem of the present invention.

As used herein, the term “periodinated” refers to a compound containing a maximum possible amount of iodine substituents while satisfying the proviso that the compound does not contain any iodine substituents that are in geminal or vicinal positions.

As used herein, the term “alkyl” includes any long or short chain, straight-chained, branched or cyclic aliphatic saturated or unsaturated hydrocarbon group or group containing a combination of these groups. The unsaturated alkyl groups may be mono- or polyunsaturated and include both alkenyl and alkynyl groups. Alkyl, alkenyl and alkynyl groups may contain up to 40 carbon atoms. However, alkyl, alkenyl and alkynyl groups containing up to 10 eg. 8, more preferably up to 6, and especially preferably up to 4 carbon atoms are preferred. Of course, the minimum number of carbons is restricted by the presence of cycles and unsaturated groups. Hence, saturated alkyl groups can have 1-40, 1-10, 1-8, 1-6 or 1-4 carbon atoms if they are linear or branched and 3-40, 3-10, 3-8, 3-6, 5-6 or 3-4 carbon atoms if they are cyclic or contain a cyclic alkyl moiety; unsaturated alkenyl or alkynyl groups can have 2-40, 2-10, 2-8, 2-6 or 2-4 carbon atoms, respectively.

The term “alkoxyl” or “alkoxy” represents —O-alkyl. In particular, the alkyl group is an alkyl group as defined hereinabove. An example of an alkoxyl is a C1-C6 alkoxyl, which represents a straight or branched alkyl chain having from one to six carbon atoms attached to an oxygen atom. Exemplary C1-C6 alkoxyl groups include methoxyl, ethoxyl, propoxyl, isopropoxyl, butoxyl, sec-butoxyl, t-butoxyl, pentoxyl, hexoxyl, and the like. C1-C6 alkoxyl includes within its definition a C1-C4 alkoxyl. Of course, the alkyl group contained in the alkoxy group may also have any other meaning specified above, such as being an unsaturated alkenyl or alkynyl group with e.g. 2 to 6 carbon atoms.

In some embodiments of the invention, the term “alkoxyalkyl” refers to an alkyl group substituted with an alkoxy group, wherein the alkyl and alkoxy groups are as defined hereinabove. An example is a C1-C6 alkyl group carrying an C1-C6 alkoxy group. The number of carbon atoms of the alkoxy group may be selected independently of the number of carbon atoms in the alkyl group.

In some embodiments of the invention, the term “hydroxyalkoxyalkyl” refers to an alkoxyalkyl group, as defined above, which is substituted at the alkoxy part with a hydroxy group. This group can be illustrated by the following structure: —R—O—R′—OH wherein R represents an alkyl group, as defined above, and R′ represents the alkyl part of an alkoxy group —O—R′ as defined above.

In some embodiments of the invention, the term “polyhydroxyalkyl” refers to an alkyl group, as defined above, carrying 2 or more hydroxy groups, e.g. 2, 3, 4, 5 or 6 hydroxy groups.

In some embodiments of the invention, the term “poly alkyleneoxyalkyl” refers to an alkyl group as defined above that is substituted with a poly alkyleneoxy group. A typical structure of a poly alkyleneoxyalkyl may thus be represented by the following formula: —R—(O—R″)ₙ—H, wherein R represents an alkyl group as defined above, R″ represents an alkylene group typically having 2, 3 or 4 and preferably 2 carbon atoms, and n is selected from the range of 2-20, preferably 2-10. The number of carbon atoms in the individual alkylene groups need not always be the same, i.e. in the above formula, the number of carbon atoms may independently be selected from 2, 3 or 4 for each of the alkylene oxy moieties —O—R″.

In some embodiments of the invention, the term “hydroxy poly alkyleneoxyalkyl” refers to a poly alkyleneoxyalkyl group as defined above that is substituted and/or terminated with a hydroxy group. A typical structure of a hydroxy poly alkyleneoxyalkyl may thus be represented by the following formula: —R—(O—R″)ₙ—OH, wherein R represents an alkyl group as defined above, R″ represents an alkylene group typically having 2, 3 or 4 and preferably 2 carbon atoms, and n is selected from the range of 2-20, preferably 2-10. The number of carbon atoms in the individual alkylene groups need not always be the same, i.e. in the above formula, the number of carbon atoms may independently be selected from 2, 3 or 4 for each of the alkylene oxy moieties —O—R″.

In some embodiments of the invention, the term “alkylcarbonyloxalkyl” represents an alkyl group as defined above that is substituted by an alkyl ester group and it typically has the following structure: —R—C(═O)—O—R′″, wherein R represents an alkyl group as defined above, typically having 1-6 carbon atoms, and R′″ represents an alkyl group as defined above, typically having 1-6 carbon atoms independently selected from the number of carbon atoms of the R group. According to another variant of the invention, it is also possible to use this kind of group, but wherein the orientation of the ester group is reversed, i.e. wherein the group is characterized by the following structure: —R—O—C(═O)—R′″, wherein R represents an alkyl group as defined above, typically having 1-6 carbon atoms, and R′″ represents an alkyl group as defined above, typically having 1-6 carbon atoms independently selected from the number of carbon atoms of the R group. Hence, according to this alternative variant, references to “alkylcarbonyloxalkyl” are to be understood as references to this alternative group with a reversed orientration of the ester group. This alternative variant is less preferred. In particular, it is believed that the above-mentioned main variant represented by the formula —R—C(═O)—O—R′″ gives rise to superior contrast enhancement properties and it is thus preferred.

The term “aryl” as used herein refers to a carbocyclic or heterocyclic, aromatic, 5-14 membered monocyclic or polycyclic ring. Exemplary aryls include phenyl, naphthyl, anthryl, phenanthryl, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl.

In some embodiments of the invention, the term “heteroaryl” is used, which refers to an aryl group, as defined hereinabove, wherein one or more, preferably 1, 2, 3 or 4, more preferably 1 or 2, ring atoms are heteroatoms independently selected from N, O and S. In some embodiments, the above-mentioned heterocyclic aryl groups are heteroaryl groups as defined herein.

In some embodiments of the invention, the term “non-aromatic heterocycle” is used, which refers to a heterocyclic 5-14 membered monocyclic or polycyclic ring having at least one ring atom that is not capable of participating in a delocalized π-electron system. Typically, they contain 1, 2, 3 or 4 heteroatoms individually selected from N, O and S. Such non-aromatic heterocycles include fully saturated heterocycles such as tetrahydrofuran and piperidine, as well as partly saturated, partly unsaturated heterocycles such as oxazoline.

In organic chemistry, a “saturated” compound is a chemical compound that has a chain of carbon atoms linked together by single bonds. Alkanes are saturated hydrocarbons. An “unsaturated” compound is a chemical compound that contains carbon-carbon double bonds or triple bonds, such as those found in alkenes or alkynes, respectively. Saturated and unsaturated compounds need not consist only of a carbon atom chain. They can form straight chain, branched chain, or ring arrangements. They can have functional groups, as well. It is in this sense that fatty acids are classified as saturated or unsaturated. The amount of unsaturation of a fatty acid can be determined by finding its iodine number.

Unsaturated compounds are those in which addition reaction can be obtained. In a chain of carbons, such as a fatty acid, a double or triple bond will cause a kink in the chain. These kinks have macro-structural implications. Unsaturated fats tend to be liquid at room temperature, rather than solid, as the kinks in the chain prevent the molecules from packing closely together to form a solid; these fats are called oils.

The term “polyhydroxy” or “polyhydric” refers to chemical compound containing two or more hydroxyl groups per molecule.

Positron emission tomography-computed tomography (known as PET-CT) is a nuclear medicine technique which combines, in a single gantry, a positron emission tomography (PET) scanner and an X-ray computed tomography (CT) scanner, to acquire images from both devices in the same scanning session. The images are combined into a single superposed (co-registered) image. Thus, functional imaging obtained by PET, which depicts the spatial distribution of metabolic or biochemical activity in the body can be precisely aligned or correlated with anatomic imaging obtained by CT scanning. Two- and three-dimensional image reconstruction may be rendered as a function of a common software and control system.

As used herein, the term “PET-CT contrast agent” characterizes an agent that enhances the signals or images of a CT scan of a PET-CT measurement. As such, a PET-CT contrast agent would also be suitable for carrying out a CT measurement. By consequence, it is typically no different from a CT contrast agent.

CT does not require the use of a contrast agent for anatomical delineation. However, a contrast agent could also be administered to patients prior to a CT scan. This is useful to highlight structures such as blood vessels that otherwise would be difficult to differentiate from their surroundings. Using a contrast agent can also help to obtain functional information about tissues. CT contrast agents are usually administered by intravenous route. Intra-arterial or intrathecal injection can also be used in some indications. Water-soluble CT contrast agents are used to visualize vascular structures and/or organs. They can also be used to diagnose tumors due to different uptake and washout dynamics from the surrounding tissues. CT contrast agent could be any molecules intended for vascular imaging including but not limited to iomeprol, ioversol, iopromide, iohexol, iodixanol, diatrizoate meglumine, metrizoate, iothalamate meglumine, iodipamide meglumine, iopramidol, ioxilan, ioxaglate, and ioversol.

“Overweight and obesity” are defined as abnormal or excessive fat accumulation that presents a risk to health. A body mass index (BMI) over 25 is considered overweight, and over 30 is obese. Obesity is a medical condition in which excess body fat has accumulated to an extent that it may have a negative effect on health. Obesity is a leading preventable cause of death worldwide, with increasing rates in adults and children.

“Diabetes” is a chronic, metabolic disease characterized by elevated levels of blood glucose (or blood sugar), which leads over time to serious damage to the heart, blood vessels, eyes, kidneys and nerves. The most common is type 2 diabetes, usually in adults, which occurs when the body becomes resistant to insulin or stops producing enough insulin.

“Non-alcoholic fatty liver disease” (NAFLD) is an umbrella term that encompasses the entire spectrum of fatty liver disease, from isolated steatosis to NASH.

NASH stands for “Non-Alcoholic SteatoHepatitis”. It can be defined as the liver manifestation of a metabolic disorder, and is the most severe form of non-alcoholic fatty liver disease (NAFLD). NASH is closely related to the triple epidemic of obesity, pre-diabetes, and diabetes but its symptoms are often silent or non-specific to NASH, making it difficult to diagnose. As a result, NASH patients can remain unaware of their condition until late stages of the disease. NASH worsens the cardiometabolic condition of patients, and is related to higher risk of death caused by cardiovascular events¹⁸.

“Cancer” is a disease characterized by involving abnormal cell growth with the potential to invade or spread to other parts of the body. The cancer referred to herein is preferably selected from the group comprising or consisting of lung, colorectal, breast, gynaecological, head and neck, oesophageal, gastric, biliary tract, follicular and medullar thyroid, and pancreatic cancers, leukaemia, melanomas, lymphomas, multiple myelomas, sarcomas, primary brain tumours, pheochromacytoma, lipoma or sarcolipoma.

In some embodiments, especially in connection with the methods of the invention involving the imaging of patients affected by metabolic disease, the term “healthy” means that a person or a patient does not suffer from a metabolic disease, preferably selected from obesity, diabetis, Non-Alcoholic Fatty Liver Disease (NAFLD) or Non-Alcoholic SteatoHepatitis (NASH). In some embodiments, especially in connection with the methods of the invention involving the imaging of patients affected by cancer, “healthy” means that a person or a patient does not suffer from cancer.

“Machine learning” is the science of getting computers to learn and act like humans do, and improve their learning over time in autonomous fashion, by feeding them data and information in the form of observations and real-world interactions. The fundamental goal of machine learning algorithms is to generalize beyond the training samples i.e. successfully interpret data that it has never ‘seen’ before.

“Deep learning” as used in the present invention is a collection of algorithms used in machine learning, used to model high-level abstractions in data through the use of model architectures, which are composed of multiple nonlinear transformations. It is part of a broad family of methods used for machine learning that are based on learning representations of data. Deep learning is a specific approach used for building and training neural networks, which are considered highly promising decision-making nodes. An algorithm is considered deep if the input data is passed through a series of nonlinearities or nonlinear transformations before it becomes output. In contrast, most modern machine learning algorithms are considered “shallow” because the input can only go only a few levels of subroutine calling.

Deep learning removes the manual identification of features in data and, instead, relies on whatever training process it has to discover the useful patterns in the input examples. This makes training the neural network easier and faster, and it can yield better results as it applied to measuring bgl.

Within deep learning, this invention uses much of but not exclusively to deep learning methods: Recurrent neural network and convolutional neural networks.

“Recurrent neural network” or “RNNs” are a recurrent neural network is a class of artificial neural network where connections between nodes form a directed graph along a sequence. This allows it to exhibit temporal dynamic behavior for a time sequence. They are especially powerful in use cases in which context is critical to predicting an outcome and are distinct from other types of artificial neural networks because they use feedback loops to process a sequence of data that informs the final output, which can also be as a sequence of data. These feedback loops allow information to persist.

In some cases, artificial neural networks process information in a single direction from input to output. These “feedforward” neural networks include convolutional neural networks that underpin image recognition systems. RNNs, on the other hand, can be layered to process information in two directions.

A “convolutional neural network” (CNN) is a type of artificial neural network used primarily in image recognition and processing that is specifically designed to process pixel data. CNNs are powerful image processing that use deep learning to perform both generative and descriptive tasks, often using machine vison that includes image and video recognition, along with recommender systems and natural language processing. This neural network has their “neurons” arranged in such a way as to cover the entire visual field avoiding the piecemeal image processing problem of traditional neural networks.

The layers of a CNN consist of an input layer, an output layer and a hidden layer that includes multiple convolutional layers, pooling layers, fully connected layers and normalization layers. The removal of limitations and increase in efficiency for image processing results in a system that is far more effective, simpler to trains limited for image processing and natural language processing.

### The Method of the First Embodiment of the Invention

As a first embodiment, the present invention provides for a method and in particular an imaging method that allows to discriminate primary tumour and/or metastases from brown and/or beige adipose tissue, by PET-CT imaging, in a human cancer patient. The method comprises the steps of:
a) administering to said human cancer patient a PET-CT contrast agent as described hereinbelow;
b) administering a ¹⁸FDG PET tracer at least 3 hours after the administration of the PET-CT contrast agent of step a),
c) performing an ¹⁸FDG PET-CT scan, to said human subject.

Unless specified otherwise, the more specific methods of the first embodiment described hereinbelow include the above steps a) to c), i.e. the main aspect of the first embodiment.

In one variant of this embodiment, the present invention provides a method for discriminating primary tumour and/or metastases from brown and/or beige adipose tissue, by PET-CT imaging, in a human cancer patient, which comprises the steps a) to c) as specified above.

Further specific aspects of the first embodiment are specified below.

(i) Identification of tissue that is neither malignant nor metastatic in a cancer patient as well as identification of malignant tumor tissue or metastasic tissue

The ¹⁸FDG PET-CT scan may be used for detecting co-localization of positive contrast enhancement of said ¹⁸FDG and said PET-CT contrast agent. Such co-localization means that the region of interest is not malignant tumour or metastasis. This information is valuable as it may influence the decision by the treating physician/oncologist to pursue and/or adapt the treatment (chemotherapy, radiotherapy, immunotherapy, surgery and/or other anticancer therapy). In some cases, it may also avoid having to repeat the PET-CT medical imaging scan and hence permit reducing the time for care/treatment and radiation exposure.

Identifying metastases is made easier thanks to the use of the PET-CT contrast agent in accordance with the invention. Tumor tissues (primary tumor and metastases) are visible through ¹⁸FDG uptake only. However, judging the presence of tumor tissues based on ¹⁸FDG uptake only is liable to false-positive determinations since brown adipose tissue (BAT) in some cases also accumulates ¹⁸FDG. However, the present invention permits to reduce this risk of false-positive determinations since BAT also accumulates the PET-CT contrast agent. Areas where both ¹⁸FDG and the PET-CT contrast agent are visible on the PET-CT scan can then be ruled out as metastases.

Determining the total volume and/or surface and localization of BAT provides further valuable information for the treating physician. However, brown adipose tissue is not always visible and/or identifiable as such on an ¹⁸FDG PET-CT scan. Additionally, BAT cannot be detected using radiotracers other than ¹⁸FDG. In the invention, the contrast of brown and beige adipose tissue is enhanced due to the presence of the PET-CT contrast agent (contrast-enhanced positive areas).

Establishment of contrast enhancement of brown and/or beige adipose tissue is preferably determined according to Hounsfield Units¹⁹, wherein the brown and/or beige adipose tissue Hounsfield Units (HU) are in the range of −70 to 100 HU and preferably in the range of −50 to 0 HU. It is known to the person skilled in the art that the final HU values will depend on the dose that was administered to the subject. The ranges specified above are valid for a dose of 0.15 g per kg body weight.

The skilled artisan can thus easily assess in all PET-CT scans the localization and volume and/or surface of contrast-enhanced positive areas (indicative of BAT) using the PET-CT contrast agent. Both parameters are predictive of the metabolic health and outcomes of the patient. By comparing the total volume and/or surface and localization of BAT present in a patient to a matched healthy volunteer (healthy person matched with respect to e.g. age, sex, body weight, ethnicity, BMI), the prognosis of the cancer patient can be established. A higher volume and/or surface of BAT is linked with a higher energy expenditure. In the case of a cancer patient, higher energy expenditure by BAT is correlated with poorer outcomes and increased tumor aggressiveness. Said higher energy expenditure can also be detected by an increased ratio of uptake of ¹⁸FDG to PET-CT contrast agent in comparison with the uptake ratio in BAT of a matched healthy volunteer. It is also linked with the development of cancer cachexia. Cancer cachexia is associated with a very poor prognosis for cancer patients. A patient with abnormal volume and/or surface and localization of BAT should get special medical attention.

In view of the above, the following additional steps are preferably carried out after performing the method steps a) to c) of the main aspect of the first embodiment as described above for identifying tissue that is neither malignant nor metastatic in a cancer patient and/or for identifying malignant or metastatic tissue:
d) identifying areas of co-localization of positive contrast enhancement of said ¹⁸FDG and said PET-CT contrast agent;
e) identifying areas of positive contrast enhancement of ¹⁸FDG but with no co-localization of positive contrast enhancement of said ¹⁸FDG and said PET-CT contrast agent;
f) assigning areas with co-localization identified in step d) as areas with no malignant tumor or metastasis and assigning areas without co-localization identified in step e) as likely areas with malignant tumor or metastasis.

It is reasonable and thus advantageous that steps a) to c) and f) are performed in the specified order. Steps d) and e) may also be performed in the reversed order or simultaneously. Moreover, if only information on areas with no malignant tumor or metastasis is desired, step e) and the corresponding part of step f) may be omitted. Likewise, if only information on areas with malignant tumor or metastasis is desired, step d) and the corresponding part of step f) may be omitted.

It will be obvious for those skilled in the art that anatomic areas known to accumulate ¹⁸FDG for physiological reasons such as brain, myocardium, urinary tract (kidney, bladder) etc. should not be assigned as tumour tissue. Additionally, the accuracy of the method can optionally be further improved by taking additional measures with respect to the tissues assigned in step f) as likely areas with malignant tumor or metastasis, to thereby exclude the possibility of ¹⁸FDG accumulation for other reasons, such as tissue affected by inflammation. It will also be obvious for those skilled in the art that the PET-CT contrast agent can be detected after administration in organs such as the GI-tract, myocardium, liver, urinary tract due to physiological reasons and/or normal metabolism so that such areas should also not be assigned as brown and/or beige adipose tissue.

The assessment of the PET-CT scan for co-localization of positive contrast enhancement of said ¹⁸FDG and said PET-CT contrast agent is carried out to discriminate primary tumour and/or metastases from brown and/or beige adipose tissue in said human cancer patient.

As an alternative to the steps a) to c) of the above main aspect of the first embodiment, the following method is provided. The method of this specific aspect is a method for discriminating primary tumour and/or metastases from brown and/or beige adipose tissue, by PET-CT imaging, in a human cancer patient that has undergone a prior administration of ¹⁸FDG PET tracer, the method comprises the steps of:
a′) administering to said human cancer patient an oral CT contrast agent, comprising an iodinated fatty acids and/or esters and/or salts and/or mixtures thereof according to general formula I as specified above, wherein the meanings of n and of the groups R₁ and R₂ are as specified above,

and wherein the administration of said oral CT contrast agent is performed at least 3 hours prior to administration of said ¹⁸FDG PET tracer;
b′) administering ¹⁸FDG to said human cancer patient according to standard practice;
c′) performing an ¹⁸FDG PET-CT scan to said human cancer patient;
d′) comparing both PET and CT scans to assess the co-localization of positive contrast enhancement of said ¹⁸FDG and said CT contrast agent so as to discriminate primary tumour and/or metastases from brown and/or beige tissue in said human cancer patient.

In another aspect, the administration of the PET-CT contrast agent prior to the PET-CT scan is adapted for the non-invasive in vivo imaging, quantification, and/or monitoring of the activity of the brown and/or beige adipose tissue (BAT) in a subject, allowing a more precise prognosis of the cancer to be treated, resulting in more adapted treatment. As an alternative to the steps a) to c) of the above main aspect of the first embodiment, the procedure of this specific aspect follows the steps of:
a) administering a PET-CT contrast agent as described hereinbelow, e.g. comprising a biocompatible formulation of iodinated fatty acids having 16-18 carbon atoms and/or esters and/or salts and/or mixtures thereof according to general formula I;
b) administering ¹⁸FDG by intravenous route, preferably according to standard practice (for example following the EANM guidelines¹⁷) and preferably at least 3 hours after the administration of the PET-CT contrast agent of step a);
c) performing the PET-CT scan;
d) comparing the PET-CT scan to that of a healthy individual.

It is reasonable and thus advantageous that steps a) to d) are performed in the specified order.

The obtained scan can be used to assess the presence of tumour and/or metastases and/or BAT (indicating the activity and/or volume and/or surface of the brown adipose tissue). In some embodiments, the obtained scans can be used separately or in conjunction to assess the presence of tumour and/or metastases (PET) and the extent of uptake of the said CT contrast agent in brown adipose tissue (indicating the activity and/or volume and/or surface of the brown adipose tissue).

Both parameters taken separately or together allow a more precise cancer prognosis than PET-CT with an ¹⁸FDG tracer alone.

Hence, it may be advantageous to combine the steps a) to d) of the above specific aspect with the following further steps:
e) determining areas with co-localization of positive contrast enhancement of said ¹⁸FDG and said PET-CT contrast agent as highly active BAT;
f) determining areas of positive contrast enhancement of said PET-CT contrast agent but absence of ¹⁸FDG as BAT;
g) assessing a prognosis of the cancer to be treated based on the results obtained in steps d), e) and f).

For example, it was shown in example 3 that assessment of the PET-CT scans for absence of ¹⁸FDG uptake but presence of contrast enhancement due to the PET-CT contrast agent as described herein allowed easy and robust detection of brown adipose tissue, increasing the sensitivity and positive predictive value of the PET-CT scan for BAT.

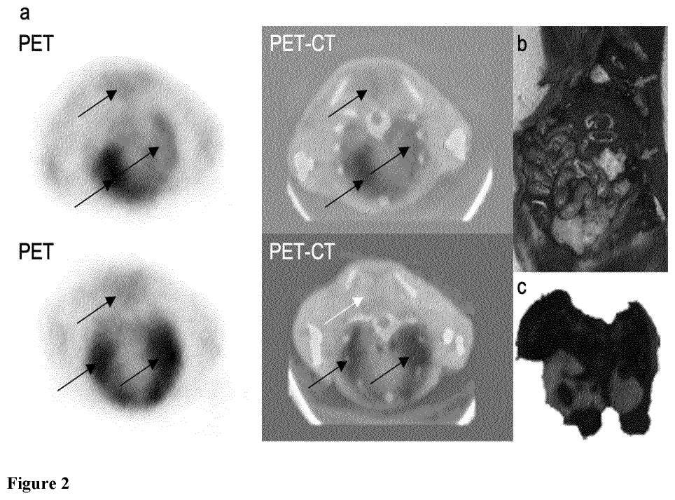

Similarly, in example 2, the ¹⁸FDG signals present in BAT, that could be interpreted as false positives (metastases), were correctly attributed to BAT, improving the specificity and positive predictive value of the PET-CT scan for tumour metastases. This was confirmed by necropsy (see FIGS. 2b, c and 3b, c).

As shown in the examples, the above described method of the invention surprisingly helps to improve the specificity and positive predictive value of the PET-CT scan for tumour metastases.

A still further object of the invention relates to the use of the PET-CT contrast agent as described hereinbelow, in a PET-CT imaging method for discriminating primary tumour and/or metastases from brown and/or beige adipose tissue, in a human cancer patient, the method comprises the steps of:
a′) administering, preferably by oral route, or optionally by other route (intravenous, intrathecal, intralymphatic, intra-arterial, intraperitoneal, or subcutaneous) to said human cancer patient said oral PET-CT contrast agent,
b′) administering an ¹⁸FDG PET tracer; at least 3 hours after the administration of the PET-CT contrast agent of step a),
c′) performing an ¹⁸FDG PET-CT scan to said human cancer patient for co-localization of positive contrast enhancement of said ¹⁸FDG and said PET-CT contrast agent.

It is reasonable and thus advantageous that steps a′) to c′) are performed in the specified order.

Assessment of the PET-CT scan for co-localization of positive contrast enhancement of said ¹⁸FDG and said PET-CT contrast agent is performed to discriminate primary tumour and/or metastases from brown and/or beige adipose tissue in said human cancer patient. Primary tumours and/or metastases are typically identified by ¹⁸FDG positive areas whereas brown and/or beige adipose tissue is typically identified by co-localization of ¹⁸FDG and PET-CT contrast agent signals or positive contrast enhancement of the PET-CT contrast agent.

(ii) Diagnosis or Staging or Re-Staging, Assessing Efficacy of Therapy or Assessing Cancer Progression

The said PET-CT scan can be prescribed for the diagnosis or staging of a cancer, to assess the efficacy of therapy, to assess cancer progression or for all reasons deemed relevant to the treating oncologist or physician. If ¹⁸FDG and the PET-CT contrast agent are colocalized, it means that the ¹⁸FDG was taken up by brown adipose tissue. On the other hand, if the ¹⁸FDG signal is not colocalized with the PET-CT contrast agent described in the present invention, ¹⁸FDG uptake by brown or beige adipose tissue can be easily and definitively ruled out. Surprisingly, this increases the specificity and positive predictive value of ¹⁸FDG PET-CT scans by reducing false positives for tumours and metastases. On the other hand, the sensitivity and the negative predictive value of ¹⁸FDG PET-CT scan for brown adipose tissue are increased by reducing false negatives.

The skilled artisan can, for example, assess both tumor progression and cancer prognosis from a single PET-CT scan using the PET-CT contrast agent described herein in accordance with the invention. As previously menitoned, the patient is first administered the PET-CT contrast agent, preferably by oral route. Then, between 3 and 72 hours, preferably 10 to 48 and more preferably 16 to 30 hours later, the patient starts the PET-CT scan procedure. A radiotracer, such as ¹⁸FDG, is injected intravenously. About 60 minutes later, the patient undergoes a PET-CT scan. The skilled artisan can get several information from this PET-CT scan. First, tumor progression. From the PET signal, the physician can for example assess if the volume and/or surface of the tumor is modified and if the number and volume and/or surface of metastases have changed in comparison with a PET signal from an earlier PET-CT scan.

In view of the above, the following additional steps are preferably carried out after performing the method steps a) to c) of the main aspect of the first embodiment as described above for cancer diagnosis:
d) identifying areas of positive contrast enhancement of ¹⁸FDG but with no co-localization of positive contrast enhancement of said ¹⁸FDG and said PET-CT contrast agent;
e) assigning areas of positive contrast enhancement of ¹⁸FDG but without co-localization of positive contrast enhancement as likely areas with malignant tumor or metastasis;
f) diagnosing the cancer by assessing localization of the areas identified in step e).

It is reasonable and thus advantageous that steps a) to f) are performed in the specified order.

As noted above, it is possible to increase the accuracy of the method by taking additional measures allowing to exclude for likely areas identified in step e) the possibility of ¹⁸FDG accumulation for other reasons, as outlined in more detail above.

In view of the above, the following additional steps are preferably carried out after performing the method steps a) to c) of the main aspect of the first embodiment as described above for tumor staging or re-staging:
d) identifying areas of positive contrast enhancement of ¹⁸FDG but with no co-localization of positive contrast enhancement of said ¹⁸FDG and said PET-CT contrast agent;
e) assigning areas of positive contrast enhancement of ¹⁸FDG but with no co-localization of positive contrast enhancement of said ¹⁸FDG and said PET-CT contrast agent as likely areas with malignant tumor or metastasis;
f) assigning the patient to the appropriate stage of the TNM system by assessing localization and volume and/or surface of the likely areas identified in step e), involvement of lymph nodes and/or presence of metastases.

It is reasonable and thus advantageous that steps a) to f) are performed in the specified order.

For re-staging of cancer, the above method comprising steps a) to f) is carried out on a patient having already been subjected earlier to cancer staging, using either the method described hereinabove or an alternative cancer staging method.

As noted above, it is possible to increase the accuracy of the method by taking additional measures allowing to exclude for likely areas identified in step e) the possibility of ¹⁸FDG accumulation for other reasons, as outlined in more detail above.

In view of the above, the following additional steps are preferably carried out after performing the method steps a) to c) of the main aspect of the first embodiment as described above for assessing efficacy of therapy or assessing cancer progression:
d) identifying areas of positive contrast enhancement of ¹⁸FDG but with no co-localization of positive contrast enhancement of said ¹⁸FDG and said PET-CT contrast agent as likely areas with malignant tumor or metastasis;
e) determining the volume and/or surface of the identified areas of positive contrast enhancement of ¹⁸FDG but without co-localization of positive contrast enhancement;
f) optionally subjecting the patient to anticancer therapy;
g) repeating steps a) to e); and
h) assigning effective therapy, if performed, and/or lack of cancer progression if the volume and/or surface of identified areas according to step g) is equal or smaller than the volume and/or surface of identified areas according to step e); and assigning lack of effectiveness of therapy, if performed, and/or cancer progression if the volume and/or surface of identified areas according to step g) is larger than the volume and/or surface of identified areas according to step e).

It is reasonable and thus advantageous that steps a) to e) are performed in the specified order. Likewise, repeated steps a) to e) as summarized under step g) are also advantageously carried out in the specified order. Step h) is to be performed as a final step. However, there is no limitation with respect to the relative order of steps d) and e) in relation to repeated steps d) and e) as summarized under step g).

As noted above, it is possible to increase the accuracy of the method by taking additional measures allowing to exclude likely areas identified in step d) and in the repetition step under item g) the possibility of ¹⁸FDG accumulation for other reasons, as outlined in more detail above.

(iii) Determination of BAT Activity and/or Cancer Prognosis

In cancer patients with highly active brown adipose tissue, characterized by the uptake of ¹⁸FDG, worse outcomes are usually observed¹³. In these patients, more metabolically active brown adipose tissue (characterized by ¹⁸FDG uptake) was associated with more active neoplastic status. Higher BAT volume and/or surface was associated with an increased likelihood of tumour recurrence and/or tumour-associated mortality¹⁴. This may be linked not only to tumour severity (inflammatory and tumour factor promoting the activation of brown adipose tissue and browning of white adipose tissue) but also to incidence of cachexia, a fatal body-wasting syndrome associated with cancer and chronic diseases.

As previously mentioned, ¹⁸FDG uptake in the brown adipose tissue is observed in about 5% of patients undergoing an ¹⁸FDG PET-CT scan. Information about the volume and/or surface of brown adipose tissue and its level of activation would give the care-givers of cancer patients precious information regarding the prognosis of the cancer. This can be achieved by using the PET-CT contrast agent described in the present invention during a planned diagnostic and/or staging PET-CT scan prescribed by the oncologist or another physician.

In view of the above, the following additional steps are preferably carried out after performing the method steps a) to c) of the main aspect of the first embodiment as described above for determining BAT activity:
d) identifying areas of co-localization of positive contrast enhancement of said ¹⁸FDG and said PET-CT contrast agent, and areas of positive contrast enhancement of said PET-CT contrast agent only;
e) determining the volume and/or surface of the areas of co-localization of positive contrast enhancement of said ¹⁸FDG and said PET-CT contrast agent as identified in step d);
f) determining the volume and/or surface of the areas of positive contrast enhancement of said PET-CT contrast agent only, as identified in step d);
g) calculating the ratio of the volume and/or surface determined in step e) divided by the volume and/or surface determined in step f);
h) comparing the ratio with a reference ratio determined as an average value by performing steps a) to g) on a population of cancer patients; and
i) assigning increased BAT activity if the determined ratio is higher than the reference ratio, assigning normal or reduced BAT activity if the determined ratio is about the same as the reference ratio and assigning reduced BAT activity if the determined ratio is lower than the reference ratio.

It is reasonable and thus advantageous that steps a) to i) are performed in the specified order except for steps e) and f), which may also be performed simultaneously or in the reversed order.

For determining cancer prognosis, the same steps a) to i) may be carried out as described above for determining BAT activity. Substequently, the following step j) may be added for obtaining a prognosis:
j) assigning poor prognosis in patients suffering from hormone-dependent cancer if reduced BAT activity is assigned in step i) and assigning poor prognosis in patients suffering from hormone-independent cancer if increased BAT activity is assigned in step i).

The addition of the PET-CT contrast agent described hereinbelow allows a more precise prognosis of cancer progression and outcome depending on the extent of contrast uptake in the brown adipose tissue and volume and/or surface of brown adipose tissue. For most malignancies, an increased uptake of ¹⁸FDG in brown adipose tissue means a poorer cancer prognosis.

In yet another specific variant of this object of the present invention, a prognostic method for cancer evaluation by assessing the quantity of active brown and/or beige adipose tissue, by PET-CT imaging, in a human cancer patient is provided. The method comprises steps a) to c) of the main aspect of the first embodiment as described above, or alternatively the steps of:
a) administering to said cancer patient a PET-CT contrast agent as described herein, wherein the administration of said PET-CT contrast agent is performed at least 3 hours prior to administration of ¹⁸FDG;
b) administering ¹⁸FDG by intravenous route, preferably according to standard practice, e.g. as described for example in literature¹⁷;
c) performing a PET-CT scan to said human cancer patient.

In this method, an uptake of the PET-CT contrast agent by brown adipose tissue may be taken as a basis for determining the prognosis of cancer. In some embodiments of the invention, uptake of the PET-CT contrast agent by brown adipose tissue may be compared with a reference value for healthy patients with matching relevant characteristics (healthy person matched with respect to e.g. age, sex, body weight, ethnicity, BMI). In most cancer types, a poor prognosis may be identified for human cancer patients having a higher uptake compared to the reference value, i.e. larger areas of positive contrast enhancement. For other malignancies (cancers influenced by hormones such as some breast cancers), higher uptake means a better cancer outcome.

In view of the above, the following additional steps are preferably carried out after performing steps a) to c) of the main aspect of the first embodiment as described above:
d) determining volume and/or surface of areas of positive contrast enhancement with co-localization of positive contrast enhancement of said ¹⁸FDG and said PET-CT contrast agent;
e) comparing determined volume and/or surface with reference value determined as an average value by performing steps a) to d) on a population of cancer patients;
f) assigning a poor prognosis in patients suffering from hormone-dependent cancer if the quantified volume and/or surface is lower than the reference value and assigning a poor prognosis in patients suffering from hormone-independent cancer if the quantified volume and/or surface is higher than the reference value.

It is reasonable and thus advantageous that steps a) to f) are to be performed in the specified order.

In another variant, and in view of the above, all BAT may be used as a basis for the prognosis. In this variant, the following additional steps are preferably carried out after performing steps a) to c) of the main aspect of the first embodiment as described above:
d) determining the volume and/or surface of positive contrast enhancement of the PET-CT contrast agent;
e) comparing the determined volume and/or surface with a reference value derived from a healthy person or group of healthy persons;
f) assigning a poor prognosis in patients suffering from hormone-dependent cancer if the quantified volume and/or surface is lower than the reference value and assigning a poor prognosis in patients suffering from hormone-independent cancer if the quantified volume and/or surface is higher than the reference value.

It is reasonable and thus advantageous that steps a) to f) are to be performed in the specified order.

The accuracy of the prognosis may be improved by combining the above steps a) to f) with further steps:
g) determining areas without co-localization of positive contrast enhancement of said ¹⁸FDG and said PET-CT contrast agent as likely tumors or metastasis;
h) assigning a prognosis based on the outcome of step f) and, additionally, based on volume and/or surface and/or localization of tumors or metastasis based on the determination of step g).

For instance, in said step h), a factor contributing to poor prognosis is a spread of the tissue identified in step g) to distant lymph nodes and/or the identification of multiple metastases.

As noted above, it is possible to increase the accuracy of the method by taking additional measures allowing to exclude likely areas identified in step g) the possibility of ¹⁸FDG accumulation for other reasons, as outlined in more detail above.

The said PET-CT scan can be prescribed for the detection, diagnosis or staging of a tumour, to assess the efficacy of therapy, to assess cancer progression or for all reasons deemed relevant to the treating oncologist or physician.

In the first embodiment, the cancer is preferably a cancer involving malignant tumors and more preferably is selected from the group comprising or consisting of lung, colorectal, breast, gynaecological, head and neck, oesophageal, gastric, biliary tract, follicular and medullar thyroid, and pancreatic cancers, leukaemia, melanomas, lymphomas, multiple myelomas, sarcomas, primary brain tumours, pheochromacytoma, lipoma or liposarcoma.

### The Method of the Second Embodiment of the Invention

Advantageously, the PET-CT contrast agent described hereinbelow can also be used in human subjects who do not have cancer but suffer from a metabolic disease such as obesity or diabetes, or in healthy volunteers.

Thus a further object of the invention is to provide a method for identifying a metabolic disease in a human subject, by PET-CT imaging brown and/or beige adipose tissue in said human subject, the method comprises the steps of:
a) administering to said human subject a PET-CT contrast agent as described hereinbelow,
b) administering an ¹⁸FDG PET tracer at least 3 hours after the administration of the PET-CT contrast agent of step a),
c) performing an ¹⁸FDG PET-CT scan to said human subject.

The presence of a metabolic disease may for instance be identified by quantifying positive contrast enhancement and comparing the determined value with a reference value for healthy individuals: if the quantified positive contrast enhancement shows a deviation from the healthy individual reference value, this may be taken as an indicator for the presence of a metabolic disease. Said healthy individual reference value may be derived by carrying out the above procedure with steps a) to c) and, if applicable, averaging the values obtained from a healthy individual or cohort of healthy individuals with matching, relevant characteristics (healthy person matched with respect to e.g. age, sex, ethnicity, body weight, BMI).

For example, uptake of PET-CT contrast agent reflects the amount of BAT present in the subject. Higher quantity of BAT is connected to lower glycemia, cholesterol (LDL), triglycerides and free fatty acids and better insulin sensitivity. By contrast, absence or low volumes and/or surfaces of BAT is linked with poor metabolic status, i.e., hyperglycaemia, high blood levels of cholesterol (LDL), triglycerides and free fatty acids and low insulin sensitivity. An obese patient with appropriate presence of BAT is in a much better metabolic state than one with low or absent BAT. The second category of patients needs special medical attention and care, including nutritional intervention to adjust their caloric intake and a personalized exercise program. They also should get additional diagnostic procedures to assess if they have NAFLD and/or NASH and appropriate treatment if required.

In view of the above, the following additional steps are preferably carried out after performing the method described above:
d) quantifying the volume and/or surface of positive contrast enhancement of the PET-CT contrast agent in the PET-CT scan of step c);
e) comparing quantified volume and/or surface of step d) with a reference value derived from one or more healthy persons;
f) assigning a poor metabolic state if the quantified volume and/or surface is lower than the reference value and a good metabolic state if the quantified volume and/or surface is equal or higher than the reference value.

It is reasonable and thus advantageous that steps a) to f) of this method are performed in the specified order.

The skilled person is able to judge the reliability of such a determination based on the degree of the deviation and the measurement error.

According to one embodiment, said human subject is suffering from obesity, type 2 diabetes, Non-alcoholic fatty liver disease (NAFLD) or Non-Alcoholic SteatoHepatitis (NASH).

According to another embodiment, said human subject is a subject not suffering from cancer.

In the above method, the reference value is preferably derived from a healthy person matched with the patient with respect to age range, sex, ethnicity, body mass range and/or BMI range.

### The Method of the Third Embodiment of the Invention

The PET-CT contrast agent described hereinbelow and the method of the invention is also envisioned to be used as a companion diagnostic in clinical or preclinical studies to help develop new therapies for metabolic diseases or to select which patients could benefit from these therapies, or to assess the efficacy of the said therapies in human or animal subjects. For example, some researchers aim to develop treatments against obesity by activating BAT and/or inducing WAT-to-BAT conversion. These researches could greatly benefit from using the contrast agent described hereinbelow to assess the efficacy of the treatment early. On the other hand, people developing anti-cancer therapies may want to screen potential study subjects to avoid patients developing cachexia or to divide the patients into subgroups and assess the treatment efficacy with or without cachexia. They could do so by performing a PET-CT scan with the contrast agent to assess the volume and/or surface of BAT in each subject during screening. Researchers developing treatments for cachexia may want to start the treatment as early as possible, ideally before weight loss. Similarly, the screening of patients could be performed using the PET-CT contrast agent and assessing the volume and/or surface of BAT in each cancer patient. In all these examples, the volunteers and/or patients would:
1) Be administered the PET-CT contrast agent as described hereinbelow, preferably by oral route;
2) At least 3 hours later, be administered ¹⁸FDG or another PET tracer;
3) Undergo a ¹⁸FDG PET-CT scan, or, if another PET tracer is used, a PET-CT scan appropriate for said other PET tracer, preferably according to standard protocol as described e.g. in literature 1.

The investigator can then assess the volume and/or surface and/or localization of BAT and compare it to that of a healthy individual with matching, relevant characteristics (healthy person matched with respect to e.g. age, sex, body weight, ethnicity, BMI). These characteristics can include, but are not limited to, sex, age, body weight, ethnicity, BMI. Volunteers and/or patients can also be used as their own control to assess the efficacy of the treatment.

Thus, when developing treatments against obesity and/or type 2 diabetes, the PET-CT scan of the patient after single or multiple adminstrations of the treatment may be compared with a PET-CT scan of the patient before administration of the treatment. Efficacy of the treatment may be confirmed if volume and/or surface of BAT has increased. An increase of the volume and/or surface of active BAT may also be indicative of efficacy and likewise an increase in the ratio of active BAT to all BAT. Alternatively, the efficacy of such treatments may be assessed by adding a placebo arm of test persons, performing the above steps 1) to 3) on the persons of the placebo group and establishing that the above-mentioned effect of BAT increase is not observed or observed to a lesser extent in the placebo group.

In view of the above, the present invention provides a first variant of the method for assessing the efficacy of a treatment against obesity and/or type 2 diabetes, which focuses on the amount of active BAT and which comprises the following steps:
1) Administering the PET-CT contrast agent as described hereinbelow prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7);
10) Comparing the volume and/or surface determined in step 8) with the volume and/or surface determined in step 9);
11) Assiging efficacy of the obesity and/or type 2 diabetes treatment if the comparison of step 10) shows an increase of volume and/or surface from step 8) to step 9).

Said method may be performed with a single patient or, preferably, with a group of patients. In the case where said method is performed with a group of patients, the results for the individual patients may be assessed together, for reducing statistical error.

In the above method, it is reasonable and thus advantageous that the steps 1) to 7) are carried out in the specified order, while steps 8) and 9) may also be performed in the reversed order or simultaneously.

For the variant relying on placebo controls, the method comprises the following steps:
1) Administering the PET-CT contrast agent as described hereinbelow, to the patient or group of patients prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow, to the patient after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7);
10) Comparing the volume and/or surface determined in step 8) with the volume and/or surface determined in step 9);
11)Performing steps 1) to 10) with a different patient or group of patients, but wherein a placebo treatment is administered in step 4 instead of the obesity and/or type 2 diabetes treatment;
12) Assiging efficacy of the obesity and/or type 2 diabetes treatment if the comparison of step 10) in the obesity and/or type 2 diabetes treatment patient or patient group shows an increase of volume and/or surface from step 8) to step 9) larger than that obtained in the comparison of step 10) in the placebo patient or patient group.

In the above method, it is reasonable and thus advantageous that the steps 1) to 7) are carried out in the specified order, while steps 8) and 9) may also be performed in the reversed order or simultaneously. The same applies to the corresponding steps summarized as step 11). The steps summarized as step 11) may be performed before, simultaneously with or after the steps 1) to 10).

The present invention further provides a second variant of the method for assessing the efficacy of a treatment against obesity and/or type 2 diabetes, which focuses on the amount of all BAT and which comprises the following steps:
1) Administering the PET-CT contrast agent as described hereinbelow prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow after administration of the obesity and/or type 2 diabetes treatment;
6) At least 3 hours later, administering ¹⁸FDG;
7) Performing a ¹⁸FDG PET-CT scan;
8) Determining the volume and/or surface of areas of positive contrast enhancement of said PET-CT contrast agent in the PET-CT scan of step 3);
9) Determining the volume and/or surface of areas of positive contrast enhancement of said PET-CT contrast agent in the PET-CT scan of step 7);
10) Comparing the volume and/or surface determined in step 8) with the volume and/or surface determined in step 9);
11) Assiging efficacy of the obesity and/or type 2 diabetes treatment if the comparison of step 10) shows an increase of volume and/or surface from step 8) to step 9).

In the above method, it is reasonable and thus advantageous that the steps 1) to 7) are carried out in the specified order, while steps 8) and 9) may also be performed in the reversed order or simultaneously.

For the variant focusing on all BAT and relying on placebo controls, the method comprises the following steps:
1) Administering the PET-CT contrast agent as described hereinbelow, to the patient or group of patients prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow, to the patient after administration of the obesity and/or type 2 diabetes treatment;
6) At least 3 hours later, administering ¹⁸FDG;
7) Performing a ¹⁸FDG PET-CT scan;
8) Determining the volume and/or surface of areas of positive contrast enhancement of said PET-CT contrast agent in the PET-CT scan of step 3);
9) Determining the volume and/or surface of areas of positive contrast enhancement of said PET-CT contrast agent in the PET-CT scan of step 7);
10) Comparing the volume and/or surface determined in step 8) with the volume and/or surface determined in step 9);
11)Performing steps 1) to 10) with a different patient or group of patients, but wherein a placebo treatment is administered in step 4 instead of the obesity and/or type 2 diabetes treatment;
12) Assiging efficacy of the obesity and/or type 2 diabetes treatment if the comparison of step 10) in the obesity and/or type 2 diabetes treatment patient or patient group shows an increase of volume and/or surface from step 8) to step 9) larger than that obtained in the comparison of step 10) in the placebo patient or patient group.

In the above method, it is reasonable and thus advantageous that the steps 1) to 7) are carried out in the specified order, while steps 8) and 9) may also be performed in the reversed order or simultaneously. The same applies to the corresponding steps summarized as step 11). The steps summarized as step 11) may be performed before, simultaneously with or after the steps 1) to 10).

The present invention further provides a third variant of the method for assessing the efficacy of a treatment against obesity and/or type 2 diabetes, which focuses on the ratio of amount of active BAT versus the amount of all BAT and which comprises the following steps: 1) Administering the PET-CT contrast agent as described hereinbelow prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7)
10)Determining the volume and/or surface of areas of positive contrast enhancement of said PET-CT contrast agent in the PET-CT scan of step 3);
11)Determining the volume and/or surface of areas of positive contrast enhancement of said PET-CT contrast agent in the PET-CT scan of step 7);
12) Calculating a first ratio by dividing the volume and/or surface determined in step 8) by the volume and/or surface in step 10);
13) Calculating a second ratio by dividing the volume and/or surface determined in step 9) by the volume and/or surface in step 11);
14) Comparing the ratio determined in step 12) with the ratio determined in step 13);
15) Assiging efficacy of the obesity and/or type 2 diabetes treatment if the comparison of step 14) shows an increase of ratio from step 12) to step 13).

In the above method, it is reasonable and thus advantageous that the steps 1) to 7) are carried out in the specified order, while steps 8) to 11) and similarly steps 12) and 13) may also be performed in the reversed order or simultaneously.

For the variant focusing on the ratio of active BAT to all BAT and relying on placebo controls, the method comprises the following steps:
1) Administering the PET-CT contrast agent as described hereinbelow, to the patient or group of patients prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow, to the patient after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7)
10)Determining the volume and/or surface of areas of positive contrast enhancement of said PET-CT contrast agent in the PET-CT scan of step 3);
11)Determining the volume and/or surface of areas of positive contrast enhancement of said PET-CT contrast agent in the PET-CT scan of step 7);
12) Calculating a first ratio by dividing the volume and/or surface determined in step 8) by the volume and/or surface in step 10);
13) Calculating a second ratio by dividing the volume and/or surface determined in step 9) by the volume and/or surface in step 11);
14) Comparing the ratio determined in step 12) with the volume and/or surface determined in step 13);
15)Performing steps 1) to 14) with a different patient or group of patients, but wherein a placebo treatment is administered in step 4 instead of the obesity and/or type 2 diabetes treatment;
16) Assiging efficacy of the obesity and/or type 2 diabetes treatment if the comparison of step 14) in the obesity and/or type 2 diabetes treatment patient or patient group shows an increase of ratio from step 12) to step 13) larger than that obtained in the comparison of step 14) in the placebo patient or patient group.

In the above method, it is reasonable and thus advantageous that the steps 1) to 7) are carried out in the specified order, while steps 8) to 11) and similarly steps 12) and 13) may also be performed in the reversed order or simultaneously. The same applies to the corresponding steps summarized as step 15). The steps summarized as step 15) may be performed before, simultaneously with or after the steps 1) to 14).

When assessing the efficacy of an anti-cancer therapy under development, a similar approach may be taken. That is, the PET-CT scan of the patient after single or multiple adminstrations of the anti-cancer treatment may be compared with a PET-CT scan of the patient before administration of the treatment. Efficacy of the treatment may be confirmed if one or more of tumor size, number of metastases and/or activity of BAT has decreased after the treatment compared to the respective parameter before the treatment. Alternatively, PET-CT scans of one or more patients after single or multiple adminstrations of the anti-cancer treatment may be compared with a PET-CT scans of one or more patients having received placebo or, preferably, reference treatment. Efficacy of the treatment may be confirmed if one or more of tumor size, number of metastases and/or activity of BAT has decreased in the test group compared to the placebo or reference group.

In view of the above, the present invention provides a first variant of the method for assessing the efficacy of an anti-cancer treatment, which comprises the following steps:
1) Administering the PET-CT contrast agent as described hereinbelow prior to administration of the anti-cancer treatment;
2) At least 3 hours later, administering ¹⁸FDG;
3) Performing a ¹⁸FDG PET-CT scan;
4) Administering the anti-cancer treatment one or more times;
5) Administering the PET-CT contrast agent as described hereinbelow after administration of the anti-cancer treatment;
6) At least 3 hours later, administering ¹⁸FDG;
7) Performing a ¹⁸FDG PET-CT scan;
8) Determining the volume and/or surface of areas of positive contrast enhancement of ¹⁸FDG but without co-localization of positive contrast enhancement in the PET-CT scan of step 3);
9) Determining the volume and/or surface of areas of positive contrast enhancement of ¹⁸FDG but without co-localization of positive contrast enhancement in the PET-CT scan of step 7);
10) Comparing the volume and/or surface determined in step 8) with the volume and/or surface determined in step 9);
11) Assigning efficacy of the anti-cancer treatment if the comparison of step 10) does not show an increase of volume and/or surface from step 8) to step 9).

Said method may be performed with a single patient or, preferably, with a group of patients. In this case, the results for the individual patients may be assessed together, for reducing statistical error.

In the above method, it is reasonable and thus advantageous that the steps 1) to 7) are carried out in the specified order, while steps 8) and 9) may also be performed in the reversed order or simultaneously.

For the variant relying on placebo or, preferably, refence treatment controls, the method comprises the following steps:
1) Administering the PET-CT contrast agent as described hereinbelow, to the patient or group of patients prior to administration of the anti-cancer treatment;
2) At least 3 hours later, administering ¹⁸FDG;
3) Performing a ¹⁸FDG PET-CT scan;
4) Administering the anti-cancer treatment one or more times;
5) Administering the PET-CT contrast agent as described hereinbelow, to the patient after administration of the anti-cancer treatment;
6) At least 3 hours later, administering ¹⁸FDG;
7) Performing a ¹⁸FDG PET-CT scan;
8) Determining the volume and/or surface of areas of positive contrast enhancement of ¹⁸FDG but without co-localization of positive contrast enhancement in the PET-CT scan of step 3);
9) Determining the volume and/or surface of areas of positive contrast enhancement of ¹⁸FDG but without co-localization of positive contrast enhancement in the PET-CT scan of step 7);
10) Comparing the volume and/or surface determined in step 8) with the volume and/or surface determined in step 9);
11)Performing steps 1) to 10) with a different patient or group of patients, but wherein a placebo treatment or reference treatment is administered in step 4 instead of the anti-cancer treatment;
12) Assiging efficacy of the anti-cancer treatment if the comparison of step 10) in the anti-cancer treatment patient or patient group shows an increase of volume and/or surface from step 8) to step 9) smaller than that obtained in the comparison of step 10) in the placebo patient or patient group or, when using a reference treatment in step 11), assigning efficacy of the anti-cancer treatment if the comparison of step 10) in the anti-cancer treatment patient or patient group shows an increase of volume and/or surface from step 8) to step 9) smaller than or equal to that obtained in the comparison of step 10) in the patient or patient group receiving the reference treatment.

In the above method, it is reasonable and thus advantageous that the steps 1) to 7) are carried out in the specified order, while steps 8) and 9) may also be performed in the reversed order or simultaneously. The same applies to the corresponding steps summarized as step 11). The steps summarized as step 11) may be performed before, simultaneously with or after the steps 1) to 10).

Aspects of Procedure Common to all Methods of the Invention

The ¹⁸FDG PET-CT scan or other PET-CT scan in step c) is preferably performed 6 to 72, more preferably 8 to 48 hours and even more preferably 16 to 30 hours after the administration of the PET-CT contrast agent of step a). The ¹⁸FDG PET-CT scan or other PET-CT scan in step c) is preferably performed 30 to 120 minutes, preferably 45 to 75 minutes, more preferably 55 to 65 minutes after the administration of ¹⁸FDG or other radiotracer of step b).

According to an embodiment, the administration of the PET-CT contrast agent occurs on the day before the planned PET-CT scan. Preferably, the administration of the PET-CT contrast agent is performed at least 3 hours, such as at least 4 hours or at least 6 hours or at least 10 hours, and especially 3 to 72 hours, more preferably 10 to 48 hours, and even more preferably 16 to 30 hours before the administration of ¹⁸FDG or other PET tracer.

Preferably, the PET-CT contrast agent is orally administered (i.e. via peroral route). While the PET-CT contrast agent is preferably given to the patient per oral route, it can also be administered by other routes (intravenous, intrathecal, intralymphatic, intra-arterial, intraperitoneal, subcutaneous).

In a preferred embodiment of the invention, the PET-CT image is sagittal or coronal. Sagittal or coronal views are preferred because it avoids the assessment of axial slices of the PET-CT scan by a radiologist and/or nuclear medicine physician which is time- and resource-consuming.

In another embodiment of the present invention, the assessment of the PET-CT scan is performed by a software or a plug-in or add-in or add-on in an existing software.

Preferably, the software analysis is performed by machine learning such as by ANN, RNN, DL or CNN techniques.

In a preferred embodiment, the method of the present invention is adapted for use in human patients, and especially human cancer patients, wherein said human patients/human cancer patients are below 18 years of age.

The Active Agent of the PET-CT Contrast Agent for Use in the Invention

The PET-CT contrast agent for use in the invention comprises as an active imaging agent one or more iodinated fatty acids and/or esters and/or salts and/or mixtures thereof. This is preferably a compound according to general formula I:

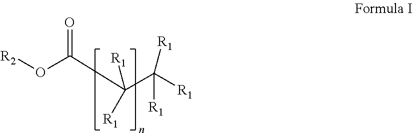

wherein n=14-16;

each R₁ is independently selected from H or I, with the provisions that the number of iodine atoms is 1 to 6, and that the iodine atoms are neither geminal nor vicinal;

and wherein R₂ is selected from H, unsaturated or saturated, linear or branched alkyl, alkoxyalkyl, hydroxyalkoxyalkyl, polyhydroxyalkyl, poly alkyleneoxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles. Each of these halogen substituents may be independently selected from F, Cl, Br and I. If multiple iodine atoms are present, the same proviso applies as defined herein for the R₁ groups, i.e. there must not be any two iodine atoms in geminal or vicinal position.

Preferably, the R₂ group may be mono or poly-substituted.

Preferable R₂ groups include but are not limited to alkyl groups such as methyl, ethyl, propyl, isopropyl, butyl, isobutyl, t-butyl, cyclopropylmethyl, pentyl, isopentyl, hexyl, isohexyl, heptyl, isoheptyl, octyl, isooctyl, 2-propenyl, allyl, crotyl, 1-butenyl, 2-butenyl, butadienyl, ethynyl, 1-propynyl, 2-propynyl, 1-butynyl, 2-butynyl, 3-butynyl and propargyl, cyclopentyl, cyclohexyl, cycloheptyl, adamantyl; aryl groups such as phenyl, naphthyl, anisyl, toluyl, xylenyl, aralkyl, aralkyloxy, heteroaryl groups such as pyrimidine, morpholine, piperazine, piperidine, and thiophene, 1-cyclohexylpropyl, or haloalkyl groups such as fluoromethyl, 1-fluoroethyl, 2-fluoroethyl, difluoromethyl, trifluoromethyl and pentafluoroethyl, chlorodimethyl, chloromethyl, 2-chloroethyl, 2,4-dichlorophenyl, 1,1,2,2-tetrachloroethyl, 1-chlorobutyl, and 4-chlorobenzyl.

It can include substituted alkyl groups such as 9-fluorenylmethyl, poly alkyleneoxyalkyl such as methoxyethoxymethyl, non-aromatic heterocycles such as tetrahydropyranyl, optionally substituted alkylcarbonyloxalkyl groups such as pivalyloxymethyl and phenylacetoxymethyl, optionally substituted arylcarbonyl groups such as phenacyl and substituted phenacyl such as p-bromophenacyl, p-methoxyphenacyl, and also substituted and unsubstituted alkyl or alkenyl groups such as t-butyl, 3-methyl-3-pentyl, cyclopentyl, cycohexyl, allyl, 3-buten-1-yl, cinnamyl, as well as heteroaryl groups such as oxazole, and 2-alkyl-1,3-oxazoline.

It can also include alkylaryl such as benzyl, substituted benzyl such as triphenylmethyl, p-methoxybenzyl, 4-picolyl, diphenylmethyl, phenylethyl, substituted phenylethyl, but also alkoxyalkyl such as methoxymethyl, ethoxymethyl, propoxymethyl, butoxymethyl, methoxyethyl, ethoxyethyl, propoxyethyl, isopropoxyethyl, butoxyethyl, isobutoxyethyl, hydroxyalkoxyalkyl such as hydroxymethoxymethyl, 2-hydroxyethoxymethyl, 3-hydroxypropoxymethyl, 4-hydroxybutoxymethyl, hydroxymethoxyethyl, hydroxymethoxypropyl, hydroxymethoxybutyl, hydroxymethoxypentyl, hydroxymethoxyhexyl, polyhydroxyalkyl, and hydroxypolyalkyleneoxyalkyl and also carbonyloxylalkyl such as acetoyloxymethyl, propanoyloxymethyl, butanoyloxymethyl, pentanoyloxymethyl, hexanoyloxymethyl, pivaloyloxymethyl, and similar groups.

The iodinated fatty acids having 16 to 18 carbon atoms and/or esters and/or salts and/or mixtures thereof according to general formula I are in some embodiments characterized by one of the following sub-formulae A, B, and C.

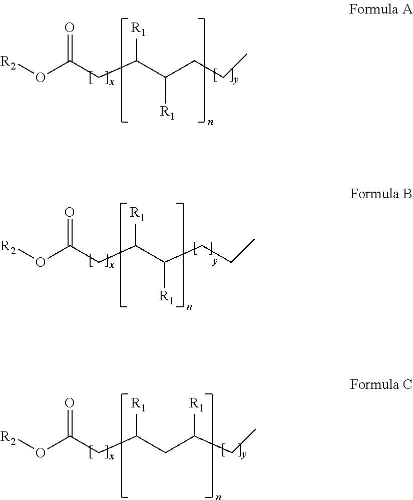

where n is an integer of 1-6, and x, y are carbon atoms in which x=0-15 and y=0-15 and x+y≤15 with the provision that the total number of carbon atoms in the fatty acid moiety of formulae A, B or C respectively is between 14 and 20, preferably between 16 and 18; and each R₁ is independently selected from H or I, with the provisions that the number of iodine atoms is 1 to 6, and that the iodine atoms are neither geminal nor vicinal; and where R₂ group is the same as specified above for formula I and may be mono or poly-substituted.

Thus suitable R₂ groups for formulae A, B or C include for example unsubstituted alkyl, such as methyl, ethyl, propyl, isopropyl, butyl, isobutyl, pentyl, isopentyl, hexyl, isohexyl, heptyl, isoheptyl, octyl, isooctyl and similar but also substituted alkyl groups such as 9-fluorenylmethyl, methoxyethoxymethyl, tetrahydropyranyl, pivalyloxymethyl, phenylacetoxymethyl, phenacyl and substituted phenacyl such as p-bromophenacyl, p-methoxyphenacyl, and also t-butyl, 3-methyl-3-pentyl, cyclopentyl, cycohexyl, allyl, 3-buten-lyl, cinnamyl, oxazole, 2-alkyl-1,3-oxazoline and similar. It also includes alkylaryl such as benzyl, substituted benzyl such as triphenylmethyl, p-methoxybenzyl, 4-picolyl, dipohenylmethyl phenylethyl, substituted phenylethyl, but also alkoxyalkyl such as methoxymethyl, ethoxymethyl, propoxymethyl, butoxymethyl, methoxyethyl, ethoxyethyl, propoxyethyl, isopropoxyethyl, butoxyethyl, isobutoxyethyl, hydroxyalkoxyalkyl such as hydroxymethoxymethyl, 2-hydroxyethoxymethyl, 3-hydroxypropoxymethyl, 4-hydroxybuthoxymethyl, hydroxymethoxyethyl, hydroxymethoxypropyl hydroxymethoxybutyl, hydroxymethoxypentyl, hydroxymethoxyhexyl, polyhydroxyalkyl, hydroxypolyalkyleneoxyalkyl, and also carbonyloxylalkyl such as acetoyloxymethyl, propanoyloxymethyl, butanoyloxymethyl, pentanoyloxymethyl, hexanoyloxymethyl, pivaloyloxymethyl, and similar groups.

The iodinated fatty acids having 16 to 18 carbon atoms and/or esters and/or mixtures thereof according to the invention may exist as isomeric mixtures or as single isomers. If not specified both isomeric forms are intended. Where a compound of the invention contains one chiral centre, the iodinated compound can be provided as a single isomer (R or S) or as a mixture of isomers, for example a racemic mixture. Where an iodinated compound of the invention contains more than one chiral centre, the iodinated compound can be provided as an enantiomerically pure diastereoisomer or as a mixture of diastereoisomers.

According to an embodiment of the invention, it is also envisioned that the PET-CT contrast agent comprises iodinated fatty acids having 4 to 24 carbon atoms and/or esters thereof that can be used in a mixture comprising several or at least two iodinated fatty acids having different carbon chains of 4 to 24 carbon atoms. In a preferred embodiment of the invention, the PET-CT contrast agent comprises iodinated fatty acids having preferably 12 to 22 carbon atoms, more preferably 14 to 20 and even more preferably 16 to 18 carbon atoms according to general formula I.

In an even more preferred embodiment, the iodinated fatty acid is an iodinated linolenic acid.

Preferably the iodinated fatty acids are periodinated.

According to a preferred embodiment, the iodinated fatty acids comprise 12 to 22, e.g. 12 to carbon atoms, preferably 14 to 20 or more preferably 14 to 18 carbon atoms and even more preferably 16 to 18 carbon atoms and/or esters and/or salts and/or mixtures thereof can be used according to the present invention.

In another embodiment, the iodinated fatty acids having 16 to 18 carbon atoms and/or esters and/or mixtures thereof according to the present invention has at least one asymmetric center. As a consequence of this asymmetric center, the iodinated compound of the present invention can occur in any of the possible stereoisomeric forms, and can be used in mixtures of stereoisomers, which can be optically active or racemic, or can be used alone as essentially pure stereoisomers, i.e., at least 95% pure. All asymmetric forms, individual stereoisomers and combinations thereof, are within the scope of the present invention.

According to yet another embodiment of the invention, the PET-CT contrast agent consists of iodinated fatty acids having 16 to 18 carbon atoms and/or esters thereof which can be used in a mixture comprising several or at least two iodinated fatty acids of 16 to 18 carbon atoms. Preferably, the PET-CT contrast agent is a biocompatible emulsion of iodinated fatty acids having 16 to 18 carbon atoms according to general formula I.

According to another embodiment, the biocompatible formulation is a formulation of ethiodized oil.

Preparation of the Active Agent of the PET-CT Contrast Agent

It is understood that any suitable method for preparing the iodinated fatty acids having 16-18 carbon atoms and/or esters and/or salt and/or mixtures thereof of formula (I) known to the skilled in the art may be encompassed by the scope of the present invention. Suitable methods of preparation are described for instance at pages 22-23 and in the Examples 1-15 of WO 2019/030024 A1 as well as pages 28-30 and Examples 1-10 of WO 2020/165349 A1.

The PET-CT Contrast Agent for Use in the Invention

The said PET-CT contrast agent is adapted for oral (i.e. peroral) route.

According to an embodiment of the invention, the PET-CT contrast agent is adapted for non-invasive in vivo imaging, quantification, and/or monitoring of the activity of the brown and/or beige adipose tissue (BAT) in a human subject.

Preferably, the PET-CT contrast agent is in the form of a biocompatible formulation. More preferably, the biocompatible formulation is an emulsion.

In accordance with the invention, the emulsion comprises biocompatible emulsifiers selected from the group comprising lecithins, polyoxyethylene sorbitan fatty acid esters, sucrose stearate, polyoxyethylene stearate, sucrose esters, sorbitan esters and/or their mixtures.

According to said embodiment, the amount of said biocompatible emulsifiers in the emulsion is between 5-50% (w/w) of the total emulsion. Preferably, the amount of said biocompatible emulsifiers in the emulsion is between 5-25% (w/w) of the total emulsion.

The amount of iodinated fatty acid and/or esters and/or salt and/or mixtures thereof of formula (I) should be at least 10% and preferably at least 20% by weight of the emulsion; a content of 30% is generally preferred, but emulsions containing up to 40% contrast agent can be prepared in some cases. One or more emulsifying agents are preferably included in the composition.

Preferably, the emulsion is a micro-emulsion or nano-emulsion.

In other embodiments, the weight of excipients may be between about 0.1% to about 75% of the total weight of the unit dose, or between about 2% to about 50% of the total weight of the unit dose, for example, and any range derivable therein.

The active agent of the PET-CT contrast agent and in particular the iodinated fatty acids having 16-18 carbon atoms and/or esters and/or salt and/or mixtures thereof of formula (I) (dissolved in an organic solvent or neat) can be added to ion-free water or buffer, preferably containing emulsifiers. The resulting mixture may be emulsified using any method known to a person skilled in the art at a temperature above the melting point of the fatty acid, to produce a finely dispersed oil-in-water emulsion. Agitation may be effected by any known means, e.g. by the use of a high shear agitator or ultrasonically. The water phase can contain other excipients such as preservatives (such as antimicrobial and/or antioxidants), stabilizers, texture-modifiers, colorants, taste-modifying agents, pharmaceutically acceptable salts and/or buffering agents. Suitable emulsions and methods of their preparation are described for instance at pages 25-27 and in Example 16 of WO 2019/030024 A1 as well as pages 32-35 and Examples 1-10 of WO 2020/165349 A1. These patent documents are incorporated herein in their entirety.

Solid pharmaceutical forms are also envisioned as the PET-CT contrast agent. In these solid forms, the active agent of the PET-CT contrast agent, as described hereinabove, is mixed with suitable excipients such as gelatin, polylactic acid, polylactic-polyglycolic acid, poloxamers, caprolactones, celluloses, sugars, sugar derivatives, salts, fatty acids and their derivatives, etc.

Dosage and Administration of the PET-CT Contrast Agent for Use in the Invention

According to a preferred embodiment of the invention, the PET-CT contrast agent is administrated at a dose corresponding to between 0.004 and 0.5 mg of iodine per gram of body weight.

However, in certain embodiments, a unit dose of the emulsion may comprise, for example, a total amount of iodine corresponding to at least about 0.004 mg of iodine per g of body weight of the PET-CT contrast agent. Likewise, in certain embodiments, a unit dose of the emulsion may comprise, for example, a total amount of iodine corresponding to about 0.5 mg or less of iodine per g of body weight of the PET-CT contrast agent.

In other non-limiting examples, a unit dose may also comprise PET-CT contrast agent in such an amount that the content of iodine is from about 1 μg per kg of body weight (hereinafter: “g/kg body weight”), about 50 μg/kg body weight, about 100 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 300 mg/kg body weight, about 350 mg/kg body weight, about 400 mg/kg body weight, about 450 mg/kg body weight, about 500 mg/kg body weight, about 600 mg/kg body weight, about 700 mg/kg body weight, about 800 mg/kg body weight, about 900 mg/kg body weight, about 1000 mg/kg body weight, about 2000 mg/kg body weight to about 5000 mg/kg body weight or more per administration, and any range derivable therein. In non-limiting examples of a derivable range from the numbers listed herein, a range of about 350 mg/kg body weight to about 1000 mg/kg body weight, about 50 μg/kg body weight to about 500 mg/kg body weight, and the like, can be administered.

In any case, the dose of the PET-CT contrast agent that is to be used depends on the particular condition being diagnosed, the severity of the condition, the individual patient parameters including age, physical condition, size and weight, the duration of the imaging, the nature of concurrent therapy (if any) and other similar factors that are within the knowledge and expertise of the health practitioner. These factors are known to those of skilled in the art and can be addressed with minimal routine experimentation. Accordingly, the optimum dosage may be determined by the practitioner who is diagnosing any particular patient.

The PET Tracer for Use in the Invention

PET scan requires the administration of a radioactive tracer (also referred to as PET tracer or radiotracer) to be performed. The most commonly used radiotracer for cancer detection is ¹⁸FDG (2-[fluorine-18]fluoro-2-deoxy-D-glucose); it is a radioactive glucose analog. The tracer is injected intravenously prior to the PET scan preferably as an isotonic, sterile, pyrogen free, clear, colorless citrate buffered solution. Said solution preferably contains, per mL, between 0.37 to 3.7 GBq (10.0-100 mCi) of 2-deoxy-2-[18F]fluoro-D glucose at the end of synthesis, 4.5 mg of sodium chloride and 7.2 mg of citrate ions. The pH of the solution is preferably between 5.0 to 7.5. The usual ¹⁸FDG dose for adults is between 185 and 370 MBq. The patient undergoing the PET-CT scan should be fasted for at least 4 hours before ¹⁸FDG adminitration. During the intraveneous injection of ¹⁸FDG and the subsequent uptake phase, the patient should remain seated or recumbent and silent to minimise ¹⁸FDG uptake in muscles. The patient should be kept warm starting 30-60 min before the injection of ¹⁸FDG and continuing throughout the subsequent uptake period and examination to minimise ¹⁸FDG accumulation in brown adipose tissue¹⁷. The PET-CT scan should be performed 55 to 75 minutes after administration of ¹⁸FDG.

Other radiotracers can also be used, such as ¹¹C-acetate, ¹¹C-methionine, ¹¹C-choline, copper(⁶⁴Cu) dotatate, ¹⁸F-EF5, ¹⁸F-fluciclovine, ¹⁸F-fluorocholine, ¹⁸F-Fluoroethyl-L-tyrosine (¹⁸F-FET), ¹⁸F-fluoromisonidazole (1⁸F-FMISO), ¹⁸F-fluorothymidine, ⁶⁴Cu—Cu-ETS2, ⁶⁸Ga-DOTA-pseudopeptides, ⁶⁸Ga-DOTA-TATE, ⁶⁸Ga-PSMA. ⁶⁸Ga—CXCR. The state of the art on such alternative PET tracers is reviewed by F. Giammarile et al.²⁰

As noted hereinabove and below, according to some variants of the methods of the present invention, it is possible to carry out the methods of the present invention, but using other PET tracers instead of ¹⁸FDG. These variants of the methods of the present invention may thus rely on the PET tracers listed above and/or discussed in the review article by Gimmarile as cited above, which is incorporated herein in its entirety.

Not all alternative PET tracers are suitable for all types of cancers. However, the skilled person is able to identify suitable alternative PET tracers for a cancer type of interest, relying on the common general knowledge as reflected for instance by the above-mentioned review article by Giammarile et al. Unless indicated otherwise or the context dictates otherwise, if another PET tracer is used instead of ¹⁸FDG, the further indications provided herein for using ¹⁸FDG, e.g. with respect to its form of administration, timing of administration, sequence of method steps, etc., may be applied in an analogous manner to the alternative PET tracer.

### Special Embodiment

The present application also pertains to the following specific embodiment. It is contemplated that the following information provided for the special embodiment of this section may be combined with information provided in other sections of this text if and to the extent such combination is technically reasonable, e.g. that the respective information to be combined relates to the same type of method. However, it is also contemplated as one distinct embodiment that the following information for the special embodiment of this section be considered independently of the information provided in other sections of this text.

Although methods and materials similar or equivalent to those described herein can be used in the practice or testing of the present special embodiment, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present special embodiment is not entitled to antedate such publication by virtue of prior invention. In addition, the materials, methods, and examples are illustrative only and are not intended to be limiting.

In the case of conflict, the present specification, including definitions, will control.

Unless defined otherwise, all technical and scientific terms used herein have the same meaning as is commonly understood by one of skill in art to which the subject matter herein belongs. As used herein, the following definitions are supplied in order to facilitate the understanding of the present special embodiment.

The term “comprise” is generally used in the sense of include, that is to say permitting the presence of one or more features or components.

As used in the specification and claims, the singular forms “a”, “an” and “the” include plural references unless the context clearly dictates otherwise.

As used herein the terms “subject” or “patient” are well-recognized in the art and are used interchangeably herein to refer to a mammal, including dog, cat, rat, mouse, monkey, cow, horse, goat, sheep, pig, camel, and, most preferably, a human. In some embodiments, the subject is a subject in need of a diagnosis or a subject with a diagnosed disease or disorder.

However, in other embodiments, the subject can be a healthy subject. The term does not denote a particular age or sex. Thus, adult and new-born subjects, whether male or female, are intended to be covered.

The term “formulation” or “pharmaceutical formulation” encompasses solid formulations such as tablets, enteric coated tablets, controlled-release tablets, sustained-release tablets, capsules and self-emulsifying pharmaceutical forms. It also encompasses liquid and semi-solid formulations such as solutions, suspensions, emulsions, topical preparations, suppositories, enemas, and parenteral formulations for injections and infusions.

The term “ethiodized oil”, is oil of natural origin and converted by organic synthetic procedures to ethyl esters of iodinated fatty acids to be used as injectable as a radio-opaque contrast agent that is used to outline structures in radiological investigations. Ethiodized oil is composed of iodine combined with ethyl esters of fatty acids of poppyseed oil, primarily as ethyl monoiodostearate and ethyl diiodostearate. Despite the precise structure is unknown it is comprised within the definition of formula I.

In chemistry, the term “geminal” used herein refers to the relationship between two atoms or functional groups that are attached to the same atom.

The related term “vicinal” refers to the relationship between two functional groups that are attached to adjacent atoms. Currently it is almost impossible to synthetize stable iodinated fatty acids and/or esters thereof having iodine atoms attached to adjacent carbon atoms (i.e. vicinal). Because of steric hindrance, those molecules are unstable and cannot be used for the purpose of the present special embodiment. However, it might be possible that in the future, the skilled in the art would find a technical solution to this problem. Thus, in case stable iodinated fatty acids and/or esters there of having iodine atoms in vicinal positions are provided, it is believed that those compounds will also be suitable in solving the technical problem of the present special embodiment.

As used herein, the term “alkyl” includes any long or short chain, straight-chained or branched aliphatic saturated or unsaturated hydrocarbon group. The unsaturated alkyl groups may be mono- or polyunsaturated and include both alkenyl and alkynyl groups. Such groups may contain up to 40 carbon atoms. However, alkyl groups containing up to 10 eg. 8, more preferably up to 6, and especially preferably up to 4 carbon atoms are preferred.

The term “alkoxyl” represents —O-alkyl. An example of an alkoxyl is a C1-C6 alkoxyl, which represents a straight or branched alkyl chain having from one to six carbon atoms attached to an oxygen atom. Exemplary C1-C6 alkoxyl groups include methoxyl, ethoxyl, propoxyl, isopropoxyl, butoxyl, sec-butoxyl, t-butoxyl, pentoxyl, hexoxyl, and the like. C1-C6 alkoxyl includes within its definition a C1-C4 alkoxyl.

The term “aryl” as used herein refers to a carbocyclic or heterocyclic, aromatic, 5-14 membered monocyclic or polycyclic ring. Exemplary aryls include phenyl, naphthyl, anthryl, phenanthryl, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl.

In organic chemistry, a “saturated” compound is a chemical compound that has a chain of carbon atoms linked together by single bonds. Alkanes are saturated hydrocarbons. An “unsaturated” compound is a chemical compound that contains carbon-carbon double bonds or triple bonds, such as those found in alkenes or alkynes, respectively. Saturated and unsaturated compounds need not consist only of a carbon atom chain. They can form straight chain, branched chain, or ring arrangements. They can have functional groups, as well. It is in this sense that fatty acids are classified as saturated or unsaturated. The amount of unsaturation of a fatty acid can be determined by finding its iodine number.

Unsaturated compounds are those in which addition reaction can be obtained. In a chain of carbons, such as a fatty acid, a double or triple bond will cause a kink in the chain. These kinks have macro-structural implications. Unsaturated fats tend to be liquid at room temperature, rather than solid, as the kinks in the chain prevent the molecules from packing closely together to form a solid; these fats are called oils.

The term “polyhydroxy” or polyhydric refers to chemical compound containing two or more hydroxyl groups per molecule.

Positron emission tomography-computed tomography (known as PET-CT) is a nuclear medicine technique which combines, in a single gantry, a positron emission tomography (PET) scanner and an X-ray computed tomography (CT) scanner, to acquire sequential images from both devices in the same session. The images are combined into a single superposed (co-registered) image. Thus, functional imaging obtained by PET, which depicts the spatial distribution of metabolic or biochemical activity in the body can be more precisely aligned or correlated with anatomic imaging obtained by CT scanning. Two- and three-dimensional image reconstruction may be rendered as a function of a common software and control system.

PET scan requires the administration of a radioactive tracer, usually ¹⁸FDG, to be performed. The tracer is injected prior to the PET scan. CT does not require the use of a contrast agent for anatomical delineation. However, a contrast agent can also be administered to patients prior to a CT scan. This is useful to highlight structures such as blood vessels that otherwise would be difficult to differentiate from their surroundings. Using a contrast agent can also help to obtain functional information about tissues. CT contrast agents are usually administered by intravenous route. Intra-arterial or intrathecal injection can also be used in some indications. Water-soluble CT contrast agents are used to visualize vascular structures and/or organs. They can also be used to diagnose tumors due to different uptake and washout dynamics from the surrounding tissues. CT contrast agent could be any molecules intended for vascular imaging including but not limited to iomeprol, ioversol, iopromide, iohexol, iodixanol, diatrizoate meglumine, metrizoate, iothalamate meglumine, iodipamide meglumine, iopramidol, ioxilan, ioxaglate, and ioversol.

It is an object of the present special embodiment to provide for a method for discriminating primary tumour and/or metastases from brown and/or beige adipose tissue, by PET-CT imaging, in a human cancer patient that has undergone a prior administration of ¹⁸FDG PET tracer, the method comprises the steps of:
a) administering to said human cancer patient an oral CT contrast agent, comprising an iodinated fatty acids and/or esters and/or salts and/or mixtures thereof according to general formula I.

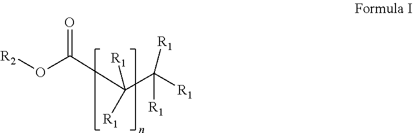

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,

and wherein the administration of said oral CT contrast agent is performed at least 3 hours prior to administration of said ¹⁸FDG PET tracer;
b) performing a PET-CT scan to said human cancer patient;
c) comparing both PET and CT scans to assess the co-localization of positive contrast enhancement of said ¹⁸FDG and said CT contrast agent so as to discriminate primary tumour and/or metastases from brown and/or beige tissue in said human cancer patient.

The said CT contrast agent is adapted for oral (i.e. peroral) route.

The said PET-CT scan can be prescribed for the diagnosis or staging of a tumour, to assess the efficacy of therapy, to assess cancer progression or for all reasons deemed relevant to the treating oncologist or physician. If both PET tracer and CT contrast agent are superimposed (located at the same location on PET and CT scans), it means that PET tracer was taken up by brown adipose tissue. On the other hand, if the PET tracer does not overlay with the CT contrast agent described in the present special embodiment, PET tracer uptake by brown or beige adipose tissue can be ruled out.

In a preferred embodiment of the special embodiment, the overlay of the PET and CT scans is based on a sagittal view. Sagittal view is preferred because it avoids the assessment of axial slices of both the PET and CT scans by a radiologist and/or nuclear medicine physician which is time-consuming.

In another embodiment of the present special embodiment, the overlay and assessment of scan overlap of the PET tracer and CT contrast agent is performed by a software or a plug-in or add-in or add-on in an existing software.

Preferably, the software analysis is performed by machine learning such as by ANN, RNN, DL or CNN techniques.

“Machine learning” is the science of getting computers to learn and act like humans do, and improve their learning over time in autonomous fashion, by feeding them data and information in the form of observations and real-world interactions. The fundamental goal of machine learning algorithms is to generalize beyond the training samples i.e. successfully interpret data that it has never ‘seen’ before.

“Deep learning” as used in the present special embodiment is a collection of algorithms used in machine learning, used to model high-level abstractions in data through the use of model architectures, which are composed of multiple nonlinear transformations. It is part of a broad family of methods used for machine learning that are based on learning representations of data. Deep learning is a specific approach used for building and training neural networks, which are considered highly promising decision-making nodes. An algorithm is considered deep if the input data is passed through a series of nonlinearities or nonlinear transformations before it becomes output. In contrast, most modern machine learning algorithms are considered “shallow” because the input can only go only a few levels of subroutine calling.

Deep learning removes the manual identification of features in data and, instead, relies on whatever training process it has to discover the useful patterns in the input examples. This makes training the neural network easier and faster, and it can yield better results as it applied to measuring bgl.

Within deep learning, this special embodiment uses much of but not exclusively to deep learning methods: Recurrent neural network and convolutional neural networks.

“Recurrent neural network” or “RNNs” are a recurrent neural network is a class of artificial neural network where connections between nodes form a directed graph along a sequence. This allows it to exhibit temporal dynamic behavior for a time sequence. The use of recurrent neural networks as a methodology in obtaining bgl is illustrated in FIG. 17. They are especially powerful in use cases in which context is critical to predicting an outcome and are distinct from other types of artificial neural networks because they use feedback loops to process a sequence of data that informs the final output, which can also be as a sequence of data. These feedback loops allow information to persist.

In some cases, artificial neural networks process information in a single direction from input to output. These “feedforward” neural networks include convolutional neural networks that underpin image recognition systems. RNNs, on the other hand, can be layered to process information in two directions.

A “convolutional neural network” (CNN) is a type of artificial neural network used primarily in image recognition and processing that is specifically designed to process pixel data. CNNs are powerful image processing that use deep learning to perform both generative and descriptive tasks, often using machine vison that includes image and video recognition, along with recommender systems and natural language processing. This neural network has their “neurons” arranged in such a way as to cover the entire visual field avoiding the piecemeal image processing problem of traditional neural networks.

The layers of a CNN consist of an input layer, an output layer and a hidden layer that includes multiple convolutional layers, pooling layers, fully connected layers and normalization layers. The removal of limitations and increase in efficiency for image processing results in a system that is far more effective, simpler to trains limited for image processing and natural language processing.

Preferably, R₂ group may be mono or poly-substituted. Suitable R₂ groups can include but are not limited to a set of alkyl substituents such as methyl, ethyl, propyl, isopropyl, butyl, isobutyl, t-butyl, cyclopropylmethyl, pentyl, isopentyl, hexyl, isohexyl, heptly, isoheptyl, octyl, isooctyl, 2-propenyl, allyl, crotyl, 1-butenyl, 2-butenyl, butadienyl, ethynyl, 1-propynyl, 2-propynyl, 1-butynyl, 2-butynyl, 3-butynyl and propagyl, cyclopentyl, cyclohexyl, cycloheptyl, admantyl; aryls substituents such as phenyl, naphthyl, anisyl, toluyl, xylenyl, aryloxy, aralkyl, aralkyloxy, heteroaryl groups (pyrimidine, morpholine, piperazine, piperidine, thiophene), 1-cyclohexylpropyl, or haloalkyls substituents such as fluoromethyl, 1-fluoroethyl, 2-fluoroethyl, difluoromethyl, trifluoromethyl and pentafluoroethyl, chlorodimethyl, chloromethyl, 2-chloroethyl, 2,4-dichlorophenyl, 1,1,2,2-tetrachloroethyl, 1-chlorobutyl, and 4-chlorobenzyl.

It can include substituted alkyl groups such as 9-fluorenylmethyl, methoxyethoxymethyl, tetrahydropyranyl, pivalyloxymethyl, phenylacetoxymethyl, phenacyl and substituted phenacyl such as p-bromophenacyl, p-methoxyphenacyl, and also t-butyl, 3-methyl-3-pentyl, cyclopentyl, cycohexyl, allyl, 3-buten-1-yl, cinnamyl, oxazole, and 2-alkyl-1,3-oxazoline.

It can also include alkylaryl such as benzyl, substituted benzyl such as triphenylmethyl, p-methoxybenzyl, 4-picolyl, dipohenylmethyl phenylethyl, substituted phenylethyl, but also alkoxyalkyl such as methoxymethyl, ethoxymethyl, propoxymethyl, butoxymethyl, methoxyethyl, ethoxyethyl, propoxyethyl, isopropoxyethyl, butoxyethyl, isobutoxyethyl, hydroxyalkoxyalkyl such as hydroxymethoxymethyl, 2-hydroxyethoxymethyl, 3-hydroxypropoxymethyl, 4-hydroxybuthoxymethyl, hydroxymethoxyethyl, hydroxymethoxypropyl hydroxymethoxybutyl, hydroxymethoxypentyl, hydroxymethoxyhexyl, polyhydroxyalkyl, and hydroxypolyalkyleneoxyalkyl and also carbonyloxylalkyl such as acetoyloxymethyl, propanoyloxymethyl, butanoyloxymethyl, pentanoyloxymethyl, hexanoyloxymethyl, pivaloyloxymethyl, and similar groups.

The iodinated fatty acids having 16 to 18 carbon atoms and/or esters and/or salts and/or mixtures thereof according to general formula I comprises the following sub-formulae A, B, and C depending on the starting material used.

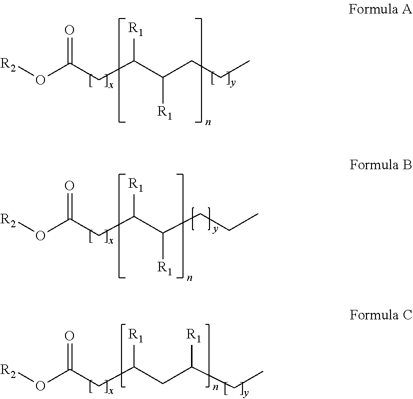

where n is an integer of 1-6, and x, y are carbon atoms in which x=0-15 and y=0-15 and x+y≤15 with the provision that the total number of carbon atoms in Formulae A, B or C respectively is ≤20; and R₁ is H or I, with the provisions that the number of iodine atoms is 1 to 6, and that the iodine atoms are neither geminal nor vicinal; and where R₂ group may be mono or poly-substituted.

Thus suitable R₂ groups include for example unsubstituted alkyl, such as methyl, ethyl, propyl, isopropyl, butyl, isobutyl, pentyl, isopentyl, hexyl, isohexyl, heptyl, isoheptyl, octyl, isooctyl and similar but also substituted alkyl groups such as 9-fluorenylmethyl, methoxyethoxymethyl, tetrahydropyranyl, pivalyloxymethyl, phenylacetoxymethyl, phenacyl and substituted phenacyl such as p-bromophenacyl, p-methoxyphenacyl, and also t-butyl, 3-methyl-3-pentyl, cyclopentyl, cycohexyl, allyl, 3-buten-lyl, cinnamyl, oxazole, 2-alkyl-1,3-oxazoline and similar. It also includes alkylaryl such as benzyl, substituted benzyl such as triphenylmethyl, p-methoxybenzyl, 4-picolyl, dipohenylmethyl phenylethyl, substituted phenylethyl, but also alkoxyalkyl such as methoxymethyl, ethoxymethyl, propoxymethyl, butoxymethyl, methoxyethyl, ethoxyethyl, propoxyethyl, isopropoxyethyl, butoxyethyl, isobutoxyethyl, hydroxyalkoxyalkyl such as hydroxymethoxymethyl, 2-hydroxyethoxymethyl, 3-hydroxypropoxymethyl, 4-hydroxybuthoxymethyl, hydroxymethoxyethyl, hydroxymethoxypropyl hydroxymethoxybutyl, hydroxymethoxypentyl, hydroxymethoxyhexyl, polyhydroxyalkyl, hydroxypolyalkyleneoxyalkyl, and also carbonyloxylalkyl such as acetoyloxymethyl, propanoyloxymethyl, butanoyloxymethyl, pentanoyloxymethyl, hexanoyloxymethyl, pivaloyloxymethyl, and similar groups.

The iodinated fatty acids having 16 to 18 carbon atoms and/or esters and/or mixtures thereof according to the special embodiment may exist as isomeric mixtures or single isomers. If not specified both isomeric forms are intended. Where a compound of the special embodiment contains one chiral centre, the iodinated compound can be provided as a single isomer (R or S) or as a mixture of isomers, for example a racemic mixture. Where an iodinated compound of the special embodiment contains more than one chiral centre, the iodinated compound can be provided as an enantiomerically pure diastereoisomer or as a mixture of diastereoisomers.

According to an embodiment of the special embodiment, it is also envisioned that the peroral contrast agent comprises iodinated fatty acids having 4 to 24 carbon atoms and/or esters thereof that can be used in a mixture comprising several or at least two iodinated fatty acids having different carbon chains of 4 to 24 carbon atoms. In a preferred embodiment of the special embodiment, the contrast agent comprises iodinated fatty acids having preferably 10 to 20 carbon atoms and more preferably 16 to 18 carbon atoms according to general formula I. In an even preferred embodiment, the iodinated fatty acid is an iodinated linolenic acid.

Preferably the iodiodinated fatty acids are periodinated.

According to a preferred embodiment, the iodinated fatty acids comprise 12 to 20 carbon atoms preferably 14 to 18 and even more preferably 16 to 18 carbon atoms and/or esters and/or salts and/or mixtures thereof can be used according to the present special embodiment.

In another embodiment, the iodinated fatty acids having 16 to 18 carbon atoms and/or esters and/or mixtures thereof according to the present special embodiment has at least one asymmetric center. As a consequence of this asymmetric center, the iodinated compound of the present special embodiment can occur in any of the possible stereoisomeric forms, and can be used in mixtures of stereoisomers, which can be optically active or racemic, or can be used alone as essentially pure stereoisomers, i.e., at least 95% pure. All asymmetric forms, individual stereoisomers and combinations thereof, are within the scope of the present special embodiment.

According to yet another embodiment of the special embodiment, the contrast agent consists of iodinated fatty acids having 16 to 18 carbon atoms and/or esters thereof which can be used in a mixture comprising several or at least two iodinated fatty acids of 16 to 18 carbon atoms. Preferably, the contrast agent is a biocompatible emulsion of iodinated fatty acids having 16 to 18 carbon atoms according to general formula I.

According to another embodiment, the biocompatible formulation is a formulation of ethiodized oil.

According to a further embodiment of the special embodiment, the biocompatible formulation of the contrast agent is an emulsion. Preferably, the emulsion is a nano-emulsion.

According to yet another embodiment of the special embodiment, the peroral contrast agent consisting in a biocompatible emulsion of iodinated fatty acids having 16 to 18 carbon atoms and/or esters thereof can be used in a mixture comprising several or at least two iodinated fatty acids.

In a preferred embodiment of the special embodiment, the contrast agent consists in a biocompatible emulsion of iodinated fatty acids having 16 to 18 carbon atoms according to general formula I.

In an even preferred embodiment, the iodinated fatty acid is an iodinated linolenic acid.

Preferably the iodinated fatty acids are periodinated.

According to yet another embodiment of the special embodiment, the contrast agent consists in a biocompatible nano-emulsion of iodinated fatty acids having 16 to 18 carbon atoms and/or esters thereof which can be used in a mixture comprising several or at least two iodinated fatty acids having different carbon chains of 16 to 18 carbon atoms. Preferably, the contrast agent is a biocompatible emulsion of iodinated fatty acids having 16 to 18 carbon atoms according to general formula I.

Solid pharmaceutical forms comprising the Computed Tomography contrast agent mixed with suitable excipients such as gelatin, polylactic acid, polylactic-polyglycolic acid, poloxamers, caprolactones, celluloses, sugars, sugar derivatives, salts, fatty acids and their derivatives, etc. are also envisioned.

In cancer patients with highly active brown adipose tissue, characterized by the uptake of ¹⁸FDG, worse outcomes are usually observed⁸. In these patients, more metabolically active brown adipose tissue (characterized by ¹⁸FDG uptake) was associated with more active neoplastic status. Higher BAT volume was associated with an increased likelihood of tumour recurrence and/or tumour-associated mortality⁹. This may be linked not only to tumour severity (inflammatory and tumour factor promoting the activation of brown adipose tissue and browning of white adipose tissue) but also to incidence of cachexia, a fatal body-wasting syndrome associated with cancer and chronic diseases.

The uptake of ¹⁸FDG and/or contrast agents in BAT visible in PET and/or CT scans respectively, is a consequence of BAT volume and/or activity.

According to an embodiment of the special embodiment, the oral contrast agent is adapted for non-invasive in vivo imaging, quantification, and/or monitoring of the activity of the brown and/or beige adipose tissue (BAT) in said human cancer patient.

Preferably, the oral contrast agent is in the form of a biocompatible formulation. More preferably, the biocompatible formulation is an emulsion.

In accordance with the special embodiment, the emulsion comprises biocompatible emulsifiers selected from the group comprising lecithins, polyoxyethylene sorbitan fatty acid esters, sucrose stearate, polyoxyethylene stearate, sucrose esters, sorbitan esters and/or their mixtures.

According to said embodiment, the amount of said biocompatible emulsifiers in the emulsion is between 5-50% (w/w) of the total emulsion. Preferably, the amount of said biocompatible emulsifiers in the emulsion is between 5-25% (w/w) of the total emulsion.

According to another embodiment of the special embodiment, the oral CT contrast agent is administrated at a dose corresponding to between 0.004 and 0.5 mg of iodine per gram of body weight.

The cancer to be diagnosed by the method is preferably selected from the group comprising or consisting of lung, colorectal, breast, gynaecological, head and neck, oesophageal, gastric, biliary tract, follicular and medullar thyroid, and pancreatic cancers, leukaemia, melanomas, lymphomas, multiple myelomas, sarcomas, primary brain tumours, pheochromacytoma, lipoma, sarcolipoma or hibernoma.

According to another embodiment, the method of the present special embodiment is adapted to cancer detection or diagnosis, and/or cancer staging and/or cancer re-staging and/or for assessing treatment performance of said cancer in said human cancer patient.

In a preferred embodiment, the method of the present special embodiment is adapted for use in human patients below 18 years of age.

As previously mentioned, ¹⁸FDG uptake in the brown adipose tissue is observed in about 5% of patients undergoing an ¹⁸FDG PET-CT scan. Information about the volume of brown adipose tissue and its level of activation would give the care-givers of cancer patients precious information regarding the prognosis of the cancer. This can be achieved by using the CT contrast agent described in the present special embodiment during a planned diagnostic and/or staging PET-CT scan prescribed by the oncologist or another physician.

In an embodiment, the administration of the CT contrast agent prior to the PET-CT scan is adapted for the non-invasive in vivo imaging, quantification, and/or monitoring of the activity of the brown and/or beige adipose tissue (BAT) in a subject, allowing a more precise prognosis of the cancer to be treated, resulting in more adapted treatment. The procedure follows the steps of:
a) administering a CT contrast agent comprising a biocompatible formulation of iodinated fatty acids having 16-18 carbon atoms and/or esters and/or salts and/or mixtures thereof according to general formula I (described above);
b) administering the PET tracer (¹⁸FDG) by intravenous route, according to standard protocol;
c) performing the PET-CT scan
d) comparing the PET-CT scan to that of a healthy individual.

The obtained scans can be used separately or in conjunction to assess the presence of tumour and/or metastases (PET) and the extent of uptake of the said CT contrast agent in brown adipose tissue (indicating the activity and/or surface and/or volume of the brown adipose tissue).

Both parameters taken separately or together allow a more precise cancer prognosis than PET-CT with an ¹⁸FDG tracer alone. The addition of this particular CT contrast agent allows a more precise prognosis of cancer progression and outcome depending on the extent of CT contrast uptake in the brown adipose tissue and volume of brown adipose tissue. For most malignancies, an increased uptake in brown adipose tissue means a poorer cancer prognosis.

The said CT contrast agent is adapted for oral (i.e. peroral) route.

The said PET-CT scan can be prescribed for the detection, diagnosis or staging of a tumour, to assess the efficacy of therapy, to assess cancer progression or for all reasons deemed relevant to the treating oncologist or physician.

According to an embodiment, the administration of the oral CT contrast agent occurs on the day before the planned PET-CT scan. Preferably, the administration of the oral CT contrast agent is performed at least 10 hours prior to administration of said ¹⁸FDG PET tracer and more preferably at least 6 hours, 4 hours and even more preferably at least 3 hours prior to administration of said ¹⁸FDG PET tracer.

According to a preferred embodiment, the peroral CT contrast agent of the method according to the special embodiment is administrated at a dose corresponding to between 0.004 and 0.5 mg of iodine per gram of body weight.

However, in certain embodiments, the nano-emulsion may comprise, for example, at least about 0.004 mg of iodine per g of body weight of the peroral CT contrast agent of the special embodiment. In other embodiments, the excipients may comprise between about 0.1% to about 75% of the weight of the unit, or between about 2% to about 30%, for example, and any range derivable therein.

In other non-limiting examples, a dose may also comprise from about 1 μg/kg/body weight, about 100 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 300 mg/kg/body weight, about 350 mg/kg/body weight, about 400 mg/kg/body weight, about 450 mg/kg/body weight, about 500 mg/kg/body weight, about 600 mg/kg/body weight, about 700 mg/kg/body weight, about 800 mg/kg/body weight, about 900 mg/kg/body weight, about 1000 mg/kg/body weight, about 2000 mg/kg/body weight to about 5000 mg/kg/body weight or more per administration, and any range derivable therein. In non-limiting examples of a derivable range from the numbers listed herein, a range of about 350 mg/kg/body weight to about 1000 mg/kg/body weight, about 50 μg/kg/body weight to about 500 mg/kg/body weight, and the like, can be administered.

In any case, the dose of the oral CT contrast agent that is to be used depends on the particular condition being diagnosed, the severity of the condition, the individual patient parameters including age, physical condition, size and weight, the duration of the imaging, the nature of concurrent therapy (if any) and other similar factors that are within the knowledge and expertise of the health practitioner. These factors are known to those of skilled in the art and can be addressed with minimal routine experimentation. Accordingly, the optimum dosage may be determined by the practitioner who is diagnosing any particular patient.

The main substrate of brown or beige adipocytes is fatty acids rather than glucose. Other imaging methods for brown or beige adipose tissue such as ¹⁸FDG-PET require activation of the brown adipose tissue, but the applicants observed that brown and beige adipose tissues can be imaged with the use of the oral CT contrast agent described in the present special embodiment without prior activation of these tissues. This is particularly advantageous in the case of a combined ¹⁸FDG PET-CT scan, in which uptake of ¹⁸FDG by off-target tissues (metabolically active, including brown adipose tissue, but non-malignant) should be avoided as much as possible.

Preparation:

It is understood that any suitable method for preparing the iodinated fatty acids having 16-18 carbon atoms and/or esters thereof of formula (I) known to the skilled in the art may be encompassed by the scope of the present special embodiment.

Emulsion:

Formulation optimization was done by experimental design. Several parameters have been assessed, including active CT ingredient, excipient(s) type and quantity, their compatibility, and method of preparation. The optimal formulation choice was based on physicochemical properties, stability and biocompatibility.

Emulsions were prepared in order to dissolve the iodinated fatty acids in water, which will improve their intestinal absorption. The emulsion formulation was improved in order to reach the fastest and the most complete absorption of the contrast agent. The aim of this last step is to reach the highest enhancement with the lower possible dose. Then, the contrast agent was tested in different conditions of brown fat activation in order to show its potential in the evaluation of the brown fat metabolism.

The following characteristics are to be achieved:
Minimal amount of emulsifiers
Low viscosity
Biocompatibility (non-toxic and non-irritating at required doses)
Prolonged stability of emulsions upon long-term storage at 4° C. or shorter term storage at 25° C.
Materials and process cost effectiveness

Lecithins, polyoxyethylene sorbitan fatty acid esters, sucrose stearate, polyoxyethylene stearate, sucrose esters and sorbitan esters surfactants are the preferred excipients used to prepare the emulsion since they have a long and documented safe use in cosmetics, food products, and pharmaceutical formulations (oral, parenteral, and topical).

The fatty acid or its derivative (dissolved in an organic solvent or neat) can be added to ion-free water or buffer, preferably containing an emulsifier, with vigorous agitation at a temperature above the melting point of the fatty acid, to produce a finely dispersed oil-in-water emulsion. Agitation may be effected by any known means, e.g. by the use of a high shear agitator or ultrasonically. The water phase can contain other excipients such as preservatives (such as antimicrobial and/or antioxidants), stabilizers, texture-modifiers, colorants, taste-modifying agents, pharmaceutically acceptable salts and/or buffering agents.

The amount of iodinated fatty acid or its derivatives should be at least 10% and preferably at least 20% by weight of the concentrated emulsion; a content of 30% is generally preferred, but emulsions as concentrated as 40% can be prepared in some cases. A small amount of an emulsifying agent is preferably included in the composition.

When an emulsifier is used, the viscosity of the emulsion will vary with the water/oil phase ratio and usually passes through a maximum value as the water/fatty acid ratio is increased. In order to obtain a fine emulsion, it is preferable to agitate for a time with the water/fatty acid ratio near to or slightly in excess of that required for maximum viscosity and then add further ion-free water with continued agitation to give the desired iodinated fatty acid concentration. The emulsion is then allowed to cool to room temperature.

Another object of the present special embodiment is to provide a prognostic method for cancer evaluation by assessing the quantity of active brown and/or beige tissue, by PET-CT imaging, in a human cancer patient that has undergone a prior administration of ¹⁸FDG PET tracer, the method comprises the steps of:
a) administering to said cancer patient an oral CT contrast agent, comprising an iodinated fatty acids and/or esters and/or salts and/or mixtures thereof according to general formula I:

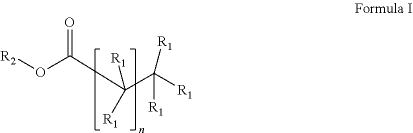

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, alkylcarbonyloxymethyl, hydroxyalkoxyalkyl, polyhydroxyalkyl, hydroxy poly alkyleneoxyalkyl,

and wherein the administration of said oral CT contrast agent is performed at least 3 hours prior to administration of said ¹⁸FDG PET tracer;
b) performing an PET-CT scan to said human cancer patient;
c) assessing the cancer progression on the PET scan and the cancer prognosis on the CT scan;

wherein, an uptake of the oral CT contrast agent by brown adipose tissue observed on the CT scan, is correlated with the prognosis of cancer patient, wherein a higher uptake compared to a healthy individual results in a poor prognosis whereas a lower uptake results in a better cancer outcome of said human cancer patient.

According to a preferred embodiment, the cancer to be diagnosed is selected from the group comprising or consisting of pheochromacytoma, lipoma, sarcolipoma,hibernoma, lung, colorectal, breast, gynaecological, head and neck, oesophageal, gastric, biliary tract, follicular and medullar thyroid, and pancreatic cancers, leukaemia, melanomas, lymphomas, multiple myelomas, sarcomas, and primary brain tumours.

In accordance with the special embodiment, the oral CT contrast agent is preferably a biocompatible emulsion of iodinated fatty acids having 16 to 18 carbon atoms according to general formula I.

Also encompassed by the present special embodiment is a method for discriminating primary tumour and/or metastases from brown and/or beige adipose tissue, by PET-CT imaging, in a human cancer patient, the method comprises the steps of:
a) administering to said human cancer patient an oral CT contrast agent, comprising an iodinated fatty acids and/or esters and/or salts and/or mixtures thereof according to general formula I:

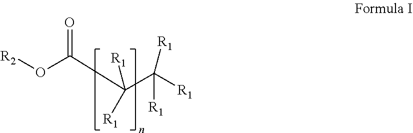

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,

and wherein the administration of said oral CT contrast agent is performed at least 3 hours prior to administration of said ¹⁸FDG PET tracer;
b) administering ¹⁸FDG to said human cancer patient by intravenous injection;
c) performing a ¹⁸FDG PET-CT scan to said human cancer patient;
d) comparing both PET and CT scans to assess the co-localization of positive contrast enhancement of said ¹⁸FDG and said CT contrast agent so as to discriminate primary tumour and/or metastases from brown and/or beige tissue in said human cancer patient.

A still further object of the special embodiment is to the use of the oral CT contrast agent comprising an iodinated fatty acids and/or esters and/or salts and/or mixtures thereof according to general formula I:

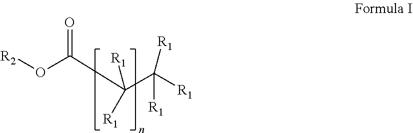

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,
in a PET-CT imaging method for discriminating primary tumour and/or metastases from brown and/or beige adipose tissue, in a human cancer patient that has undergone a prior administration of ¹⁸FDG PET tracer, the method comprises the steps of:
a) performing a ¹⁸FDG PET-CT scan to said human cancer patient;
b) comparing both PET and CT scans to assess the co-localization of positive contrast enhancement of said ¹⁸FDG and said CT contrast agent so as to discriminate primary tumour and/or metastases from brown and/or beige tissue in said human cancer patient,

with the proviso that said CT oral contrast agent fo use is administered at least 3 hours prior to administration of said ¹⁸FDG PET tracer.

Another object of the present special embodiment is to provide a prognostic method for detecting and/or assessing cancer in a patient by adding more information from one single CT or dual energy CT (DECT) scan, said method comprises the combined administration of the oral CT contrast agent described in herein (Formula I) with a water-soluble CT contrast agent administered by parenteral route to a patient suspected of developing cancer. The CT or DECT is performed as follows:
1) Oral administration of the CT contrast agent according to Formula I;
2) Parenteral administration of the water soluble radiocontrast agent;
3) Performing the CT or DECT scan on said patient.

Wherein, the uptake of the oral CT contrast agent by brown adipose tissue is correlated with the prognosis for the cancer patient, depending on cancer type. For most cancers, higher uptake than normal means a poor prognosis and additional medical attention for this patient is required.

Both objects of this particular embodiment (detection of malignant tissues and assessment of cancer prognosis) can be performed at the same time, on the same CT or DECT scan.

Dual energy CT (DECT), also known as spectral CT, is a computed tomography technique that uses two separate x-ray photon energy spectra, allowing the interrogation of materials that have different attenuation properties at different energies. Whereas conventional single energy CT produces a single image set, dual energy data (attenuation values at two energy spectra) can be used to reconstruct numerous image types.

Water-soluble CT radiocontrast agents that can be used are selected from the following non-exhaustive list:
1. High osmolarity contrast media—diatrizoate sodium/meglumine (Gastrografin, MD-Gastroview, Cystografin), metrizoate (Isopaque) and iothalamate sodium/meglumine (Conray, Cysto-Conray)
2. Low osmolarity contrast media—iopamidol (Isovue), iohexol (Omnipaque), iomeprol (Imeron), iopromide (Ultravist), ioversol (Optiray), ioxilan (Oxilan), iodixanol (Visipaque), ioxaglate (Hexabrix), iodixanol (Visipaque) and others.

Thus it is an object of the present special embodiment to combine the administration of the oral CT contrast agent described herein with the administration of a parenteral, water-soluble CT radiocontrast agent to diagnose and/or stage a tumour, and/or assess the efficacy of treatment, and/or assess cancer progression, and/or assess cancer prognosis and/or for all reasons deemed relevant to the treating oncologist or physician. The imaging modality is CT or DECT. This imaging modality can also be further combined with an ¹⁸FDG PET scan, as described above. The addition of the oral CT contrast agent described in the present special embodiment can help predicting the development of cachexia in the patient. It can also serve as a prognosis of cancer progression, as described above.

Another object of the present special embodiment to provide for a method for discriminating primary tumour and/or metastases from brown and/or beige adipose tissue, by PET-DECT imaging, in a human cancer patient that has undergone a prior administration of ¹⁸FDG PET tracer, the method comprises the steps of:
a) administering to said human cancer patient an oral CT contrast agent, comprising an iodinated fatty acids and/or esters and/or salts and/or mixtures thereof according to general formula I:

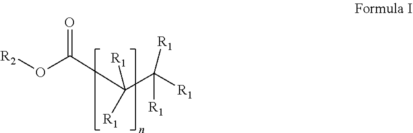

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,

and wherein the administration of said oral CT contrast agent is performed at least 3 hours prior to administration of said ¹⁸FDG PET tracer;
b) performing a ¹⁸FDG PET-DECT scan to said human cancer patient;
c) comparing both PET and DECT scans to assess the co-localization of positive contrast enhancement of said ¹⁸FDG and said oral CT contrast agent so as to discriminate primary tumour and/or metastases from brown and/or beige tissue in said human cancer patient.

Those skilled in the art will appreciate that the special embodiment described herein is susceptible to variations and modifications other than those specifically described. It is to be understood that the special embodiment includes all such variations and modifications without departing from the spirit or essential characteristics thereof. The special embodiment also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the special embodiment being indicated by the appended Claims, and all changes which come within the meaning and range of equivalency are intended to be embraced therein.

The foregoing description will be more fully understood with reference to the following Examples. Such Examples, are, however, exemplary of methods of practicing the present special embodiment and are not intended to limit the scope of the special embodiment.

The present special embodiment further relates to the following numbered items:
1. A method for discriminating primary tumour and/or metastases from brown and/or beige adipose tissue, by PET-CT imaging, in a human cancer patient that has undergone a prior administration of ¹⁸FDG PET tracer, the method comprises the steps of:
   a) administering to said human cancer patient an oral CT contrast agent, comprising an iodinated fatty acids and/or esters and/or salts and/or mixtures thereof according to general formula I:

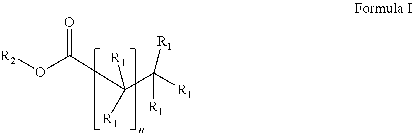

wherein n=14-16;

R₁ is H or I, with the provisions that the number of iodine atoms is 1 to 6, and that the iodine atoms are neither geminal nor vicinal;

and where R2 is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,

and wherein the administration of said oral CT contrast agent is performed at least 3 hours prior to administration of said ¹⁸FDG PET tracer;
b) performing a ¹⁸FDG PET-CT scan to said human cancer patient;
c) comparing both PET and CT scans to assess the co-localization of positive contrast enhancement of said ¹⁸FDG and said CT contrast agent so as to discriminate primary tumour and/or metastases from brown and/or beige adipose tissue in said human cancer patient.
2. The method for discriminating primary tumour and/or metastases from brown and/or beige adipose tissue according to item 1, wherein the oral CT contrast agent is adapted for non-invasive in vivo imaging, quantification, and/or monitoring of the activity of the brown and/or beige adipose tissue (BAT) in said human cancer patient.
3. The method for discriminating primary tumour and/or metastases from brown and/or beige adipose tissue according to any of items 1-2, wherein the oral CT contrast agent is in the form of a biocompatible formulation
4. The method for discriminating primary tumour and/or metastases from brown and/or beige adipose tissue according to any of items 1-3, wherein the biocompatible formulation is an emulsion.
5. The method for discriminating primary tumour and/or metastases from brown and/or beige adipose tissue according to item 4, wherein the emulsion comprises biocompatible emulsifiers selected from the group comprising lecithins, polyoxyethylene sorbitan fatty acid esters, sucrose stearate, polyoxyethylene stearate, sucrose esters, sorbitan esters and/or their mixtures.
6. The method for discriminating primary tumour and/or metastases from brown and/or beige adipose tissue according to item 5, wherein the amount of said biocompatible emulsifiers in the emulsion is between 3-50% (w/w) of the total emulsion.
7. The method for discriminating primary tumour and/or metastases from brown and/or beige adipose tissue according to item 5, wherein the amount of said biocompatible emulsifiers in the emulsion is between 5-25% (w/w) of the total emulsion.
8. The method for discriminating primary tumour and/or metastases from brown and/or beige adipose tissue according to any of items 1-7, wherein the oral CT contrast agent is administrated at a dose corresponding to between 0.004 and 0.5 mg of iodine per gram of body weight.
9. The method for discriminating primary tumour and/or metastases from brown and/or beige adipose tissue according to any of items 1-7, wherein the oral CT contrast agent is administrated at a dose corresponding to between 0.02 and 0.2 mg of iodine per gram of body weight.
10. The method for discriminating primary tumour and/or metastases from brown and/or beige adipose tissue according to any of items 1-9, wherein said cancer is selected from the group consisting of lung, colorectal, breast, gynaecological, head and neck, oesophageal, gastric, biliary tract, follicular and medullar thyroid, and pancreatic cancer, leukaemia, melanoma, lymphoma, multiple myeloma, sarcoma, pheochromacytoma and primary brain tumours.
11. The method for discriminating primary tumour and/or metastases from brown and/or beige adipose tissue according to any of items 1-10, wherein the method is adapted for use in human subjects below 18 years of age.
12. The method for discriminating primary tumour and/or metastases from brown and/or beige adipose tissue according to any of items 1-11, wherein the method is adapted to cancer detection, and/or cancer staging and/or cancer re-staging and/or for assessing treatment performance of said cancer in said human cancer patient.
13. A prognostic method for cancer evaluation by assessing the quantity and/or volume and/or activity of brown and/or beige adipose tissue, by PET-CT imaging, in a human cancer patient that has undergone a prior administration of ¹⁸FDG PET tracer, the method comprises the steps of:
a) administering to said cancer patient an oral CT contrast agent, comprising an iodinated fatty acids and/or esters and/or salts and/or mixtures thereof according to general formula I:

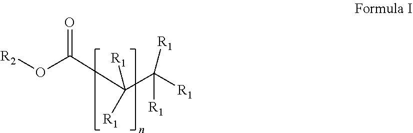

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, alkylcarbonyloxymethyl, hydroxyalkoxyalkyl, polyhydroxyalkyl, hydroxy poly alkyleneoxyalkyl,

and wherein the administration of said oral CT contrast agent is performed at least 3 hours prior to administration of said ¹⁸FDG PET tracer;
b) performing an ¹⁸FDG PET-CT scan to said human cancer patient;
c) assessing the cancer progression on the PET scan and the cancer prognosis on the CT scan;

characterized in that, an uptake of the oral CT contrast agent by brown adipose tissue observed on the CT scan, is correlated with the prognosis of cancer patient, wherein a higher uptake compared to a healthy individual results in a poor prognosis whereas a lower uptake results in a better cancer outcome of said human cancer patient.
14. The prognostic method for cancer evaluation by assessing the quantity of active brown and/or beige adipose tissue according to item 12, wherein cancer is selected from the group consisting of pheochromacytoma, lung, colorectal, breast, gynaecological, head and neck, oesophageal, gastric, biliary tract, follicular and medullar thyroid, and pancreatic cancers, leukaemia, melanomas, lymphomas, multiple myelomas, sarcomas, and primary brain tumours.
15. The prognostic method for cancer evaluation according to any of items 12-14, wherein the oral CT contrast agent is a biocompatible emulsion of iodinated fatty acids having 16 to 18 carbon atoms according to general Formula I.
16. A method for discriminating primary tumour and/or metastases from brown and/or beige adipose tissue, by PET-CT imaging, in a human cancer patient, the method comprises the steps of:
a) administering to said human cancer patient an oral CT contrast agent, comprising an iodinated fatty acids and/or esters and/or salts and/or mixtures thereof according to general formula I:

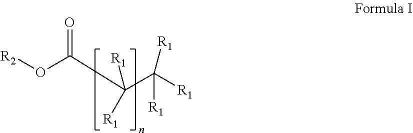

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,

and wherein the administration of said oral CT contrast agent is performed at least 3 hours prior to administration of said ¹⁸FDG PET tracer;
b) administering ¹⁸FDG to said human cancer patient by intravenous injection;
c) performing a ¹⁸FDG PET-CT scan to said human cancer patient;
d) comparing both PET and CT scans to assess the co-localization of positive contrast enhancement of said ¹⁸FDG and said CT contrast agent so as to discriminate primary tumour and/or metastases from brown and/or beige tissue in said human cancer patient.
17. Use of an oral CT contrast agent comprising an iodinated fatty acids and/or esters and/or salts and/or mixtures thereof according to general formula I:

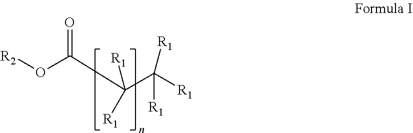

wherein n=14-16;

R₁ is H or I, with the provisions that the number of iodine atoms is 1 to 6, and that the iodine atoms are neither geminal nor vicinal;

and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, or alkylcarbonyloxalkyl,
in a PET-CT imaging method for discriminating primary tumour and/or metastases from brown and/or beige adipose tissue, in a human cancer patient that has undergone a prior administration of ¹⁸FDG PET tracer, the method comprises the steps of:
a) performing a ¹⁸FDG PET-CT scan to said human cancer patient;
b) comparing both PET and CT scans to assess the co-localization of positive contrast enhancement of said ¹⁸FDG and said CT contrast agent so as to discriminate primary tumour and/or metastases from brown and/or beige tissue in said human cancer patient,

with the proviso that said CT oral contrast agent fo use is administered at least 3 hours prior to administration of said ¹⁸FDG PET tracer.

General Remark

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

The foregoing description will be more fully understood with reference to the following Examples. Such Examples, are, however, exemplary of methods of practicing the present invention and are not intended to limit the scope of the invention.

### EXAMPLES

### Example 1: Uptake of ¹⁸FDG in Brown Adipose Tissue Leads to Potentially Erroneous Results of PET-CT Scans

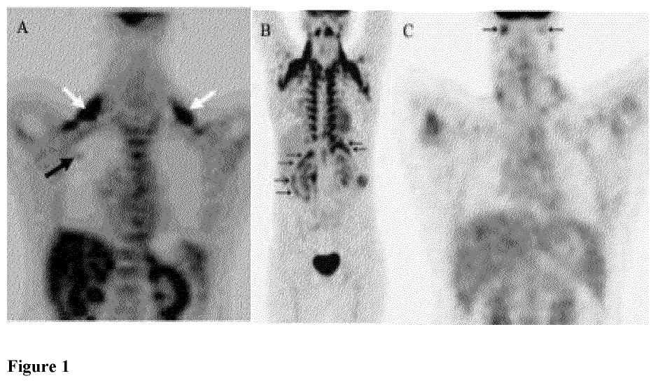

Patients undergoing a PET scan are injected ¹⁸FDG to image tumors and/or metastases. ¹⁸FDG uptake can occur in brown adipose tissue, leading to erroneous results of the PET-CT medical scans (false positives and false negatives) and/or having to repeat the medical scan. FIG. 1 shows examples of ¹⁸FDG uptake in brown adipose tissue in human cancer patients. In FIG. 1A, ¹⁸FDG uptake in BAT is higher than in the primary tumor which could lead to false negative result of PET scan for the lung cancer patient. In FIG. 1, a paediatric cancer patient with very high BAT volume and/or activity is presented. Such high BAT volume and/or activity can lead to false positive and/or false negative PET-CT scan results. In such cases, the patient is usually required to repeat the medical imaging procedure. FIG. 1C presents a case of asymmetric ¹⁸FDG uptake in BAT. In this case, the scan can lead to a false positive result of tumor and/or metastases in the neck.

### Example 2: Improved Specificity and Positive Predictive Value of ¹⁸FDG PET-CT Scan for Cancer Metastases

Syngeneic mouse melanoma model (with tumor metastases dissemination to the lungs) was induced by intravenous injection of B16F10 murine melanoma cancer cells in C57B1/6 mice. 19 days after cells injection, the animals were administered with ¹⁸FDG and a PET-CT scan was performed on each animal. One day later, the PET-CT contrast agent was administered via oral route to the same animals. Then, a second ¹⁸FDG PET-CT scan was performed. The animals were then euthanized and necropsy was performed on each animal.

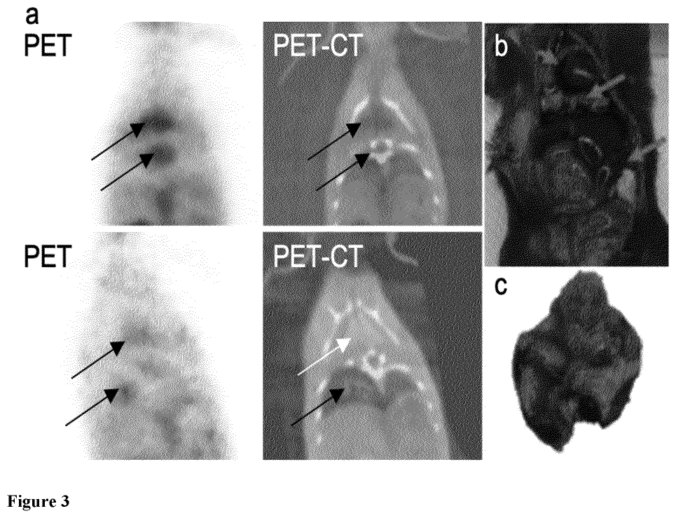

Assessment of the PET-CT scans for co-localization of positive contrast enhancement of ¹⁸FDG and PET-CT contrast agent allowed discriminating metastases from brown adipose tissue (FIGS. 2a and 3a). The ¹⁸FDG signals present in BAT, that could be interpreted as false positives (metastases), were correctly attributed to BAT, improving the specificity and positive predictive value of the PET-CT scan for tumour metastases. This was confirmed by necropsy (FIGS. 2b, c and 3b, c).

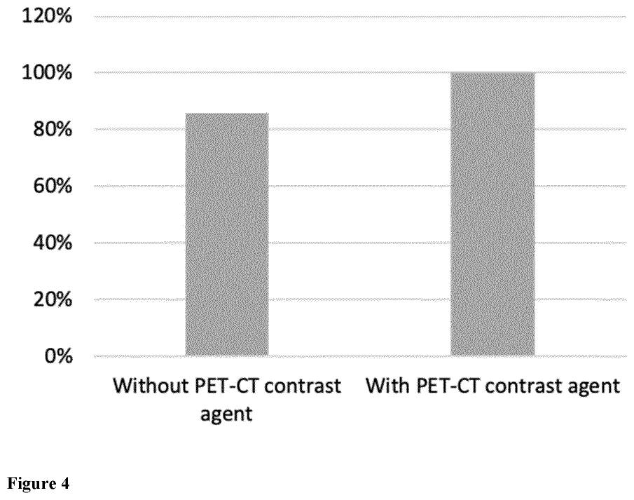

In total, based on necropsy (standard of truth), the six mice presented 24 metastases (four per mouse on average). On the ¹⁸FDG PET-CT scans, four mice had a positive signal in BAT, that could be interpreted as metastases (false positives). Without the PET-CT contrast agent, the positive predictive value PPV of the ¹⁸FDG PET-CT scan for metastases was 85% (FIG. 4). By using the PET-CT contrast agent, the PPV was 100%. The CT contrast agent thus improves the PPV of the ¹⁸FDG PET-CT scan by 15%.

### Example 3: Improved Sensitivity and Negative Predictive Value of ¹⁸FDG PET-CT Scan for Brown Adipose Tissue

Syngeneic mouse melanoma model (with tumor metastases dissemination to the lungs) was induced by intravenous injection of B16F10 murine melanoma cancer cells in C57B1/6 mice. 19 days after cells injection, the animals were administered with ¹⁸FDG and a PET-CT scan was performed on each animal. One day later, the PET-CT contrast agent was administered via oral route to the same animals. Then, a second ¹⁸FDG PET-CT scan was performed. In total, 12 ¹⁸FDG PET-CT scans were performed (six with and six without the PET-CT contrast agent). The animals were then euthanized and necropsy was performed.

Assessment of the PET-CT scans for absence of ¹⁸FDG uptake but presence of contrast enhancement due to the PET-CT contrast agent allowed easy and robust detection of brown adipose tissue, increasing the sensitivity and positive predictive value of the PET-CT scan or BAT.

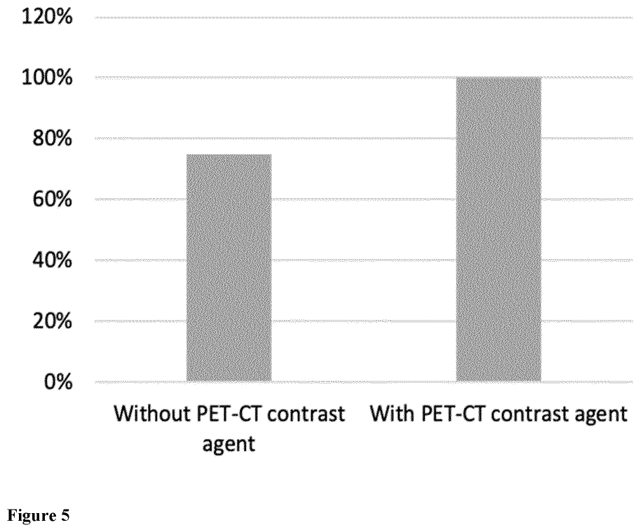

In total, ¹⁸FDG uptake in BAT was present in nine out of 12 PET-CT scans. However, all mice had BAT. Based on ¹⁸FDG uptake, the sensitivity of the PET-CT scan for BAT was 75% (FIG. 5). Based on the uptake of the PET-CT contrast agent, the sensitivity for BAT was 100%. Using the PET-CT contrast agent, the sensitivity of the PET-CT scan for BAT in mice was thus improved by 25%.

### REFERENCES

1. Al-Bulushi, N. K., Bailey, D. & Mariani, G. The Medical Case for a Positron Emission Tomography and X-ray Computed Tomography Combined Service in Oman. Sultan Qaboos Univ. Med. J. 13, 491-501 (2013).
2. Alavi, A. et al. Positron emission tomography imaging in nonmalignant thoracic disorders. Semin. Nucl. Med. 32, 293-321 (2002).
3. Carter, K. & Kotlyarov, E. Common causes of false positive F18 FDG PET/CT scans in oncology. Braz. Arch. Biol. Technol.—BRAZARCH BIOL TECHNOL 50, (2007).
4. Cypess, A. M. et al. Identification and Importance of Brown Adipose Tissue in Adult Humans. N. Engl. J. Med. 360, 1509-1517 (2009).
5. Frankl, J., Sherwood, A., Clegg, D. J., Scherer, P. E. & Öz, O. K. Imaging Metabolically Active Fat: A Literature Review and Mechanistic Insights. Int. J. Mol. Sci. 20, (2019).
6. Hong, T. S. et al. Brown adipose tissue 18F-FDG uptake in pediatric PET/CT imaging. Pediatr. Radiol. 41, 759-768 (2011).
7. Steinberg, J. D., Vogel, W. & Vegt, E. Factors influencing brown fat activation in FDG PET/CT: a retrospective analysis of 15,000+ cases. Br. J. Radiol. 90, 20170093 (2017).
8. Cohade, C., Osman, M., Pannu, H. K. & Wahl, R. L. Uptake in supraclavicular area fat (‘USA-Fat’): description on 18F-FDG PET/CT. J. Nucl. Med. Off Publ. Soc. Nucl. Med. 44, 170-176 (2003).
9. Long, N. M. & Smith, C. S. Causes and imaging features of false positives and false negatives on 18F-PET/CT in oncologic imaging. Insights Imaging 2, 679-698 (2011).
10. Wang, X. & Koch, S. Positron emission tomography/computed tomography potential pitfalls and artifacts. Curr. Probl. Diagn. Radiol. 38, 156-169 (2009).
11. Kolodny, G. & Williams, G. Method of Reducing Interferences in Positron Emission Tomography. (2008).
12. Allemann, E., Babic, A. & Vinet, L. Nanoemulsion of Iodinated Fatty Acids for Ct Imaging. (2019).
13. Huang, Y.-C. et al. The relationship between brown adipose tissue activity and neoplastic status: an (18)F-FDG PET/CT study in the tropics. Lipids Health Dis. 10, 238 (2011).
14. Chu, K., Bos, S. A., Gill, C. M., Torriani, M. & Bredella, M. A. Brown adipose tissue and cancer progression. Skeletal Radiol. 49, 635-639 (2020).
15. Bos, S. A., Gill, C. M., Martinez-Salazar, E. L., Torriani, M. & Bredella, M. A. Preliminary investigation of brown adipose tissue assessed by PET/CT and cancer activity. Skeletal Radiol. 48, 413-419 (2019).
16. Leitner, B. P. et al. Mapping of human brown adipose tissue in lean and obese young men. Proc. Natd. Acad. Sci. U.S.A 114, 8649-8654 (2017).
17. Boellaard, R. et al. FDG PET/CT: EANM procedure guidelines for tumour imaging: version 2.0. Eur. J. Nucl. Med. Mol. Imaging 42, 328-354 (2015).
18. European Association for the Study of the Liver (EASL), European Association for the Study of Diabetes (EASD), & European Association for the Study of Obesity (EASO). EASL-EASD-EASO Clinical Practice Guidelines for the management of non-alcoholic fatty liver disease. J. Hepatol. 64, 1388-1402 (2016).
19. DenOtter, T. D. & Schubert, J. Hounsfield Unit. in StatPearls (StatPearls Publishing, 2021); and Hounsfield unit | Radiology Reference Article | Radiopaedia.org. https://radiopgaedia.org/articles/hounsfield-unit.
20. Giammarile, F. et al. Non-FDG PET/CT in Diagnostic Oncology: a pictorial review. Eur. J. Hybrid Imaging 3, 20 (2019).

1. A PET-CT imaging method and especially a method for discriminating primary tumour and/or metastases from brown and/or beige adipose tissue, by PET-CT imaging, in a human subject and especially a human cancer patient, the method comprises the steps of:
a) administering to said human subject and especially human cancer patient a PET-CT contrast agent, comprising an agent selected from iodinated fatty acids and/or esters and/or salts and/or mixtures thereof according to general formula I: 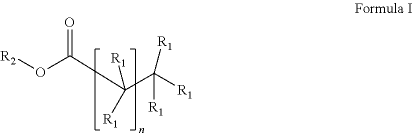
wherein n=14-16;
R₁ is H or I, with the provisions that the number of iodine atoms is 1 to 6, and that the iodine atoms are neither geminal nor vicinal;
and where R₂ is H, unsaturated or saturated, linear or branched alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles, wherein each of these halogen substituents may be independently selected from F, Cl, Br and I, wherein, if multiple iodine atoms are present, there must not be any two iodine atoms in geminal or vicinal position,
b) administering a ¹⁸FDG PET tracer; at least 3 hours after the administration of the PET-CT contrast agent of step a),
c) performing an ¹⁸FDG PET-CT scan to said human subject and especially human cancer patient.

2. Method according to claim 1, which is a method for identifying tissue that is neither malignant nor metastatic in a cancer patient and/or for identifying malignant or metastatic tissue, wherein the following steps are carried out after performing the method steps a) to c) of claim 1:
d) identifying areas of co-localization of positive contrast enhancement of said ¹⁸FDG and said PET-CT contrast agent;
e) identifying areas of positive contrast enhancement of ¹⁸FDG but with no co-localization of positive contrast enhancement of said ¹⁸FDG and said PET-CT contrast agent;
f) assigning areas with co-localization identified in step d) as areas with no malignant tumor or metastasis and assigning areas without co-localization identified in step e) as likely areas with malignant tumor or metastasis.

3. Method according to claim 1, which is a method for assessing cancer prognosis, wherein the following steps are carried out after performing the method steps a) to c) of claim 1:
d) comparing the PET-CT scan to that of a healthy individual.
e) determining areas with co-localization of positive contrast enhancement of said ¹⁸FDG and said PET-CT contrast agent as highly active BAT;
f) determining areas of positive contrast enhancement of said PET-CT contrast agent but absence of ¹⁸FDG as BAT;
g) assessing a prognosis of the cancer to be treated based on the results obtained in steps d), e) and f).

4. Method according to claim 1, which is a method for cancer diagnosis, wherein the following additional steps are carried out after performing the method steps a) to c) of claim 1:
d) identifying areas of positive contrast enhancement of ¹⁸FDG but with no co-localization of positive contrast enhancement of said ¹⁸FDG and said PET-CT contrast agent;
e) assigning areas of positive contrast enhancement of ¹⁸FDG but without co-localization of positive contrast enhancement as likely areas with malignant tumor or metastasis;
f) diagnosing the cancer by assessing localization of the areas identified in step e).

5. Method according to claim 1, which is a method for tumor staging or re-staging, wherein the following additional steps are carried out after performing the method steps a) to c) of claim 1:
d) identifying areas of positive contrast enhancement of ¹⁸FDG but with no co-localization of positive contrast enhancement of said ¹⁸FDG and said PET-CT contrast agent;
e) assigning areas of positive contrast enhancement of ¹⁸FDG but with no co-localization of positive contrast enhancement of said ¹⁸FDG and said PET-CT contrast agent as likely areas with malignant tumor or metastasis;
f) assigning the patient to the appropriate stage of the TNM system by assessing localization and volume and/or surface of the likely areas identified in step e), involvement of lymph nodes and/or presence of metastases.

6. Method according to claim 1, which is a method for assessing efficacy of therapy or assessing cancer progression, wherein the following additional steps are carried out after performing the method steps a) to c) of claim 1:
d) identifying areas of positive contrast enhancement of ¹⁸FDG but with no co-localization of positive contrast enhancement of said ¹⁸FDG and said PET-CT contrast agent as likely areas with malignant tumor or metastasis;
e) determining the volume and/or surface of the identified areas of positive contrast enhancement of ¹⁸FDG but without co-localization of positive contrast enhancement;
f) optionally subjecting the patient to anticancer therapy;
g) repeating steps a) to e); and
h) assigning effective therapy, if performed, and/or lack of cancer progression if the volume and/or surface of identified areas according to step g) is equal or smaller than the volume and/or surface of identified areas according to step e); and assigning lack of effectiveness of therapy, if performed, and/or cancer progression if the volume and/or surface of identified areas according to step g) is larger than the volume and/or surface of identified areas according to step e).

7. Method according to claim 1, which is a method for determining BAT activity, wherein the following additional steps are carried out after performing the method steps a) to c) of claim 1:
d) identifying areas of co-localization of positive contrast enhancement of said ¹⁸FDG and said PET-CT contrast agent, and areas of positive contrast enhancement of said PET-CT contrast agent only;
e) determining the volume and/or surface of the areas of co-localization of positive contrast enhancement of said ¹⁸FDG and said PET-CT contrast agent as identified in step d);
f) determining the volume and/or surface of the areas of positive contrast enhancement of said PET-CT contrast agent only, as identified in step d);
g) calculating the ratio of the volume and/or surface determined in step e) divided by the volume and/or surface determined in step f);
h) comparing the ratio with a reference ratio determined as an average value by performing steps a) to g) on a population of cancer patients; and
i) assigning increased BAT activity if the determined ratio is higher than the reference ratio, assigning normal or reduced BAT activity if the determined ratio is about the same as the reference ratio and assigning reduced BAT activity if the determined ratio is lower than the reference ratio.

8. Method according to claim 7, which is a method for cancer prognosis, wherein the following additional step is carried out after performing the method steps a) to i) of claim 7:
j) assigning poor prognosis in patients suffering from hormone-dependent cancer if reduced BAT activity is assigned in step i) and assigning poor prognosis in patients suffering from hormone-independent cancer if increased BAT activity is assigned in step i).

9. Method according to claim 1, which is a method for cancer prognosis, wherein the following additional steps are carried out after performing the method steps a) to c) of claim 1:
d) determining volume and/or surface of areas of positive contrast enhancement with co-localization of positive contrast enhancement of said ¹⁸FDG and said PET-CT contrast agent;
e) comparing determined volume and/or surface with reference value determined as an average value by performing steps a) to d) on a population of cancer patients;
f) assigning a poor prognosis in patients suffering from hormone-dependent cancer if the quantified volume and/or surface is lower than the reference value and assigning a poor prognosis in patients suffering from hormone-independent cancer if the quantified volume and/or surface is higher than the reference value.

10. Method according to claim 1, which is a method for cancer prognosis, wherein the following additional steps are carried out after performing the method steps a) to c) of claim 1:
d) determining the volume and/or surface of positive contrast enhancement of the PET-CT contrast agent;
e) comparing the determined volume and/or surface with a reference value derived from a healthy person or group of healthy persons;
f) assigning a poor prognosis in patients suffering from hormone-dependent cancer if the quantified volume and/or surface is lower than the reference value and assigning a poor prognosis in patients suffering from hormone-independent cancer if the quantified volume and/or surface is higher than the reference value.

11. Method according to claim 1, which is a method for identifying a metabolic disease in a human subject by PET-CT imaging brown and/or beige adipose tissue in said human subject, the method compring the steps a) to c) of claim 1 and the following additional steps:
d) quantifying the volume and/or surface of positive contrast enhancement of the PET-CT contrast agent in the PET-CT scan of step c);
e) comparing quantified volume and/or surface of step d) with a reference value derived from one or more healthy persons;
f) assigning a poor metabolic state if the quantified volume and/or surface is lower than the reference value and a good metabolic state if the quantified volume and/or surface is equal or higher than the reference value.

12. Method for assessing the efficacy of a treatment against obesity and/or type 2 diabetes, which comprises the following steps:
1) Administering the PET-CT contrast agent specified in claim 1 prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7);
10) Comparing the volume and/or surface determined in step 8) with the volume and/or surface determined in step 9);
11) Assiging efficacy of the obesity and/or type 2 diabetes treatment if the comparison of step 10) shows an increase of volume and/or surface from step 8) to step 9).

13. Method for assessing the efficacy of a treatment against obesity and/or type 2 diabetes, which comprises the following steps:
1) Administering the PET-CT contrast agent as described hereinbelow, to the patient or group of patients prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow, to the patient after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7);
10) Comparing the volume and/or surface determined in step 8) with the volume and/or surface determined in step 9);
11)Performing steps 1) to 10) with a different patient or group of patients, but wherein a placebo treatment is administered in step 4 instead of the obesity and/or type 2 diabetes treatment;
12) Assiging efficacy of the obesity and/or type 2 diabetes treatment if the comparison of step 10) in the obesity and/or type 2 diabetes treatment patient or patient group shows an increase of volume and/or surface from step 8) to step 9) larger than that obtained in the comparison of step 10) in the placebo patient or patient group.

14. Method for assessing the efficacy of a treatment against obesity and/or type 2 diabetes, which comprises the following steps:
1) Administering the PET-CT contrast agent as described hereinbelow prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow after administration of the obesity and/or type 2 diabetes treatment;
6) At least 3 hours later, administering ¹⁸FDG;
7) Performing a ¹⁸FDG PET-CT scan;
8) Determining the volume and/or surface of areas of positive contrast enhancement of said PET-CT contrast agent in the PET-CT scan of step 3);
9) Determining the volume and/or surface of areas of positive contrast enhancement of said PET-CT contrast agent in the PET-CT scan of step 7);
10) Comparing the volume and/or surface determined in step 8) with the volume and/or surface determined in step 9);
11) Assiging efficacy of the obesity and/or type 2 diabetes treatment if the comparison of step 10) shows an increase of volume and/or surface from step 8) to step 9).

15. Method for assessing the efficacy of a treatment against obesity and/or type 2 diabetes, which comprises the following steps:
1) Administering the PET-CT contrast agent as described hereinbelow, to the patient or group of patients prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow, to the patient after administration of the obesity and/or type 2 diabetes treatment;
6) At least 3 hours later, administering ¹⁸FDG;
7) Performing a ¹⁸FDG PET-CT scan;
8) Determining the volume and/or surface of areas of positive contrast enhancement of said PET-CT contrast agent in the PET-CT scan of step 3);
9) Determining the volume and/or surface of areas of positive contrast enhancement of said PET-CT contrast agent in the PET-CT scan of step 7);
10) Comparing the volume and/or surface determined in step 8) with the volume and/or surface determined in step 9);
11)Performing steps 1) to 10) with a different patient or group of patients, but wherein a placebo treatment is administered in step 4 instead of the obesity and/or type 2 diabetes treatment;
12) Assiging efficacy of the obesity and/or type 2 diabetes treatment if the comparison of step 10) in the obesity and/or type 2 diabetes treatment patient or patient group shows an increase of volume and/or surface from step 8) to step 9) larger than that obtained in the comparison of step 10) in the placebo patient or patient group.

16. Method for assessing the efficacy of a treatment against obesity and/or type 2 diabetes, which comprises the following steps:
1) Administering the PET-CT contrast agent as described hereinbelow prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7)
10)Determining the volume and/or surface of areas of positive contrast enhancement of said PET-CT contrast agent in the PET-CT scan of step 3);
11)Determining the volume and/or surface of areas of positive contrast enhancement of said PET-CT contrast agent in the PET-CT scan of step 7);
12) Calculating a first ratio by dividing the volume and/or surface determined in step 8) by the volume and/or surface in step 10);
13) Calculating a second ratio by dividing the volume and/or surface determined in step 9) by the volume and/or surface in step 11);
14) Comparing the ratio determined in step 12) with the ratio determined in step 13);
15) Assiging efficacy of the obesity and/or type 2 diabetes treatment if the comparison of step 14) shows an increase of ratio from step 12) to step 13).

17. Method for assessing the efficacy of a treatment against obesity and/or type 2 diabetes, which comprises the following steps:
1) Administering the PET-CT contrast agent as described hereinbelow, to the patient or group of patients prior to administration of the obesity and/or type 2 diabetes treatment;
2) At least 3 hours later, administering ¹⁸FDG;
3) Performing a ¹⁸FDG PET-CT scan;
4) Administering the obesity and/or type 2 diabetes treatment one or more times;
5) Administering the PET-CT contrast agent as described hereinbelow, to the patient after administration of the obesity and/or type 2 diabetes treatment;
6) At least 3 hours later, administering ¹⁸FDG;
7) Performing a ¹⁸FDG PET-CT scan;
8) Determining the volume and/or surface of areas of co-localization of positive contrast enhancement of said ¹⁸FDG and said PET-CT contrast agent in the PET-CT scan of step 3);
9) Determining the volume and/or surface of areas of co-localization of positive contrast enhancement of said ¹⁸FDG and said PET-CT contrast agent in the PET-CT scan of step 7)
10)Determining the volume and/or surface of areas of positive contrast enhancement of said PET-CT contrast agent in the PET-CT scan of step 3);
11)Determining the volume and/or surface of areas of positive contrast enhancement of said PET-CT contrast agent in the PET-CT scan of step 7);
12) Calculating a first ratio by dividing the volume and/or surface determined in step 8) by the volume and/or surface in step 10);
13) Calculating a second ratio by dividing the volume and/or surface determined in step 9) by the volume and/or surface in step 11);
14) Comparing the ratio determined in step 12) with the volume and/or surface determined in step 13);
15)Performing steps 1) to 14) with a different patient or group of patients, but wherein a placebo treatment is administered in step 4 instead of the obesity and/or type 2 diabetes treatment;
16) Assiging efficacy of the obesity and/or type 2 diabetes treatment if the comparison of step 14) in the obesity and/or type 2 diabetes treatment patient or patient group shows an increase of ratio from step 12) to step 13) larger than that obtained in the comparison of step 14) in the placebo patient or patient group.

18. Method for assessing the efficacy of an anti-cancer treatment, which comprises the following steps:
1) Administering the PET-CT contrast agent as described hereinbelow prior to administration of the anti-cancer treatment;
2) At least 3 hours later, administering ¹⁸FDG;
3) Performing a ¹⁸FDG PET-CT scan;
4) Administering the anti-cancer treatment one or more times;
5) Administering the PET-CT contrast agent as described hereinbelow after administration of the anti-cancer treatment;
6) At least 3 hours later, administering ¹⁸FDG;
7) Performing a ¹⁸FDG PET-CT scan;
8) Determining the volume and/or surface of areas of positive contrast enhancement of ¹⁸FDG but without co-localization of positive contrast enhancement in the PET-CT scan of step 3);
9) Determining the volume and/or surface of areas of positive contrast enhancement of ¹⁸FDG but without co-localization of positive contrast enhancement in the PET-CT scan of step 7);
10) Comparing the volume and/or surface determined in step 8) with the volume and/or surface determined in step 9);
11) Assigning efficacy of the anti-cancer treatment if the comparison of step 10) does not show an increase of volume and/or surface from step 8) to step 9).

19. Method for assessing the efficacy of an anti-cancer treatment treatment, which comprises the following steps:
1) Administering the PET-CT contrast agent as described hereinbelow, to the patient or group of patients prior to administration of the anti-cancer treatment;
2) At least 3 hours later, administering ¹⁸FDG;
3) Performing a ¹⁸FDG PET-CT scan;
4) Administering the anti-cancer treatment one or more times;
5) Administering the PET-CT contrast agent as described hereinbelow, to the patient after administration of the anti-cancer treatment;
6) At least 3 hours later, administering ¹⁸FDG;
7) Performing a ¹⁸FDG PET-CT scan;
8) Determining the volume and/or surface of areas of positive contrast enhancement of ¹⁸FDG but without co-localization of positive contrast enhancement in the PET-CT scan of step 3);
9) Determining the volume and/or surface of areas of positive contrast enhancement of ¹⁸FDG but without co-localization of positive contrast enhancement in the PET-CT scan of step 7);
10) Comparing the volume and/or surface determined in step 8) with the volume and/or surface determined in step 9);
11)Performing steps 1) to 10) with a different patient or group of patients, but wherein a placebo treatment or reference treatment is administered in step 4) instead of the anti-cancer treatment;
12) Assiging efficacy of the anti-cancer treatment if the comparison of step 10) in the anti-cancer treatment patient or patient group shows an increase of volume and/or surface from step 8) to step 9) smaller than that obtained in the comparison of step 10) in the placebo patient or patient group or, when using a reference treatment in step 11), assigning efficacy of the anti-cancer treatment if the comparison of step 10) in the anti-cancer treatment patient or patient group shows an increase of volume and/or surface from step 8) to step 9) smaller than or equal to that obtained in the comparison of step 10) in the patient or patient group receiving the reference treatment.

20. The method according to anyone of claims 1 to 19, wherein the PET-CT contrast agent is orally administered.

21. The method according to claim 20, wherein the orally administered PET-CT contrast agent is adapted for non-invasive in vivo imaging, quantification, and/or monitoring of the activity of the brown and/or beige adipose tissue (BAT) in said human cancer patient.

22. The method according to any of claims 20-21, wherein the orally administered PET-CT contrast agent is in the form of a biocompatible formulation.

23. The method according to any of claims 1-22, wherein the biocompatible formulation is an emulsion.

24. The method according to claim 23, wherein the emulsion comprises biocompatible emulsifiers selected from the group comprising lecithins, polyoxyethylene sorbitan fatty acid esters, sucrose stearate, polyoxyethylene stearate, sucrose esters, sorbitan esters and/or their mixtures.

25. The method according to claim 24, wherein the amount of said biocompatible emulsifiers in the emulsion is between 3-50% (w/w) of the total emulsion.

26. The method according to claim 24 or 25, wherein the amount of said biocompatible emulsifiers in the emulsion is between 5-25% (w/w) of the total emulsion.

27. The method according to any of claims 20-26, wherein the orally administered PET-CT agent is administrated at a dose corresponding to between 0.004 and 0.5 mg of iodine per gram of body weight.

28. The method according to any of claims 20-26, wherein the orally administered PET-CT agent is administrated at a dose corresponding to between 0.02 and 0.2 mg of iodine per gram of body weight.

29. The method according to any of claims 1-10 and 20-28, wherein said cancer is selected from the group consisting of lung, colorectal, breast, gynaecological, head and neck, oesophageal, gastric, biliary tract, follicular and medullar thyroid, and pancreatic cancer, leukaemia, melanoma, lymphoma, multiple myeloma, sarcoma, pheochromacytoma lipoma, liposarcoma and primary brain tumour.

30. The method according to any of claims 1-10 and 20-29, wherein the method is adapted for use in human cancer patients below 18 years of age.

31. The method according to any of claims 1 and 20-30, wherein the method is adapted to cancer detection, and/or cancer staging and/or cancer re-staging and/or for assessing treatment performance of said cancer in said human cancer patient.

32. A method for identifying a metabolic disease in a human subject, by PET-CT imaging brown and/or beige adipose tissue in said human subject, the method comprises the steps of:
a) administering to said human subject a PET-CT contrast agent, comprising an iodinated fatty acids and/or esters and/or salts and/or mixtures thereof according to general formula I: 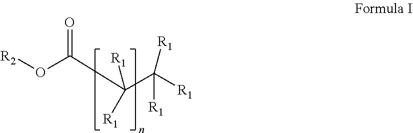
wherein n=14-16;
R₁ is H or I, with the provisions that the number of iodine atoms is 1 to 6, and that the iodine atoms are neither geminal nor vicinal;
and where R₂ is H, unsaturated or saturated, linear or branched alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles, wherein each of these halogen substituents may be independently selected from F, Cl, Br and I, wherein, if multiple iodine atoms are present, there must not be any two iodine atoms in geminal or vicinal position,
b) administering a ¹⁸FDG PET tracer or another PET tracer; at least 3 hours after the administration of the PET-CT contrast agent of step a),
c) performing an ¹⁸FDG PET-CT scan or PET-CT scan appropriate for said other PET tracer to said human subject.

33. The method for identifying a metabolic disease according to claim 32, wherein the PET-CT contrast agent is orally administered.

34. The method for identifying a metabolic disease according to any of claims 32-33, wherein the human subject is suffering from obesity, diabetes, Non-alcoholic fatty liver disease (NAFLD) or Non-Alcoholic SteatoHepatitis (NASH).

35. The method for identifying a metabolic disease according to any of claims 32-34, wherein the human subject does not have cancer.

36. Use of a PET-CT contrast agent comprising an iodinated fatty acids and/or esters and/or salts and/or mixtures thereof according to general formula I. 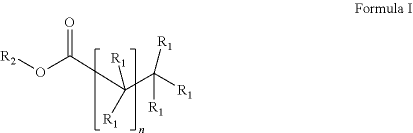
wherein n=14-16;
R₁ is H or I, with the provisions that the number of iodine atoms is 1 to 6, and that the iodine atoms are neither geminal nor vicinal;
and where R₂ is H, unsaturated or saturated, linear or branched alkyls, alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles, wherein each of these halogen substituents may be independently selected from F, Cl, Br and I, wherein, if multiple iodine atoms are present, there must not be any two iodine atoms in geminal or vicinal position,
in a PET-CT imaging method for discriminating primary tumour and/or metastases from brown and/or beige adipose tissue, in a human cancer patient, the method comprises the steps of:
a) orally administering to said human cancer patient said oral PET-CT contrast agent,
b) administering a ¹⁸FDG PET tracer; at least 3 hours after the administration of the PET-CT contrast agent of step a),
c) performing an ¹⁸FDG PET-CT scan to said human cancer patient for co-localization of positive contrast enhancement of said ¹⁸FDG and said PET-CT contrast agent.

37. A pharmaceutical composition comprising an iodinated fatty acid and/or ester and/or salt and/or mixtures thereof according to general formula I: 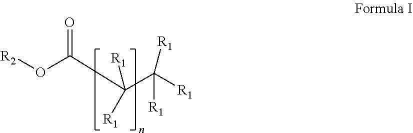
wherein n=14-16;
R₁ is H or I, with the provisions that the number of iodine atoms is 1 to 6, and that the iodine atoms are neither geminal nor vicinal;
and where R₂ is H, unsaturated or saturated, linear or branched alkyls, alkoxyalkyl, hydroxyalkoxyalkyl, polyhydroxyalkyl, hydroxy poly alkyleneoxyalkyl, aryl, aryloxy, arylcarbonyl, arylcarbonylalkyl, heteroaryl, non-aromatic heterocycle or alkylcarbonyloxalkyl, wherein these groups may be substituted by one or more, preferably 1-5 and more prefereably 1, 2, 3 or 4 substituents, each independently selected from aryl groups, heteroaryl groups, halogen, hydroxy, alkyl groups, alkoxy groups, aryloxy groups and non-aromatic heterocycles, wherein each of these halogen substituents may be independently selected from F, Cl, Br and I, wherein, if multiple iodine atoms are present, there must not be any two iodine atoms in geminal or vicinal position,
for use in cancer detection, and/or cancer staging and/or cancer re-staging and/or for assessing performance of cancer treatment in a human cancer patient and/or for identifying a metabolic disease and/or for development of treatments against obesity, diabetes type 2 or cancer and/or for use in a method as specified in any of claims 1 to 36.

38. A pharmaceutical composition comprising ¹⁸FDG for use in cancer detection, and/or cancer staging and/or cancer re-staging and/or for assessing performance of cancer treatment in a human cancer patient and/or for identifying a metabolic disease and/or for development of treatments against obesity, diabetes type 2 or cancer and/or for use in a method as specified in any of claims 1 to 36.
